SEQUENCE LISTING (56 sequences)

SEQ ID NO: 1 - DNA, 21 bp, Artificial Sequence
  Synthetic nucleic acid sequence
ctaccaggaatggccttgtcc

SEQ ID NO: 2 - DNA, 21 bp, Artificial Sequence
  Synthetic nucleic acid sequence
ctctcaggtctggtgtcatcc

SEQ ID NO: 3 - DNA, 30 bp, Artificial Sequence
  Synthetic nucleic acid sequence
gattagcatacttagactactacctccatg

SEQ ID NO: 4 - DNA, 27 bp, Artificial Sequence
  Synthetic nucleic acid sequence
gatcaacttctgaaaaagcattcccac

SEQ ID NO: 5 - DNA, 21 bp, Artificial Sequence
  Synthetic nucleic acid sequence
tcgtgagaatgtcttcccatt

SEQ ID NO: 6 - DNA, 29 bp, Artificial Sequence
  Synthetic nucleic acid sequence
tcttggattgatttgagataagtgaaatc

SEQ ID NO: 7 - DNA, 30 bp, Artificial Sequence
  Synthetic nucleic acid sequence
acgttggatggcttgttaaccagctttgcc

SEQ ID NO: 8 - DNA, 30 bp, Artificial Sequence
  Synthetic nucleic acid sequence
acgttggatgtttttcagactggcagagcg

SEQ ID NO: 9 - DNA, 30 bp, Artificial Sequence; identical to SEQ ID NO: 8
  Synthetic nucleic acid sequence

SEQ ID NO: 10 - DNA, 30 bp, Artificial Sequence; identical to SEQ ID NO: 7
  Synthetic nucleic acid sequence

SEQ ID NO: 11 - DNA, 30 bp, Artificial Sequence
  Synthetic nucleic acid sequence
acgttggatgttgctggcacccaatggaag

SEQ ID NO: 12 - DNA, 30 bp, Artificial Sequence
  Synthetic nucleic acid sequence
acgttggatgatgagagacatgacgatgcc

SEQ ID NO: 13 - DNA, 30 bp, Artificial Sequence
  Synthetic nucleic acid sequence
acgttggatgtggtggacatggtgaatgac

SEQ ID NO: 14 - DNA, 30 bp, Artificial Sequence
  Synthetic nucleic acid sequence
acgttggatgtggtgcagatgctcacatag

SEQ ID NO: 15 - DNA, 30 bp, Artificial Sequence
  Synthetic nucleic acid sequence
acgttggatgcacagagagagtctggacac

SEQ ID NO: 16 - DNA, 30 bp, Artificial Sequence
  Synthetic nucleic acid sequence
acgttggatgctcttggtctttccctcatc

SEQ ID NO: 17 - DNA, 30 bp, Artificial Sequence
  Synthetic nucleic acid sequence
acgttggatgcctctgatcctctttgcttc

SEQ ID NO: 18 - DNA, 30 bp, Artificial Sequence
  Synthetic nucleic acid sequence
acgttggatgaagagggagtggaagggaag

SEQ ID NO: 19 - DNA, 30 bp, Artificial Sequence
  Synthetic nucleic acid sequence
acgttggatgacagctctgcattcagcacg

SEQ ID NO: 20 - DNA, 30 bp, Artificial Sequence
  Synthetic nucleic acid sequence
acgttggatgagtcaatccctttggtgctc

SEQ ID NO: 21 - DNA, 30 bp, Artificial Sequence
  Synthetic nucleic acid sequence
acgttggatgactgaagctccacaatttgg

SEQ ID NO: 22 - DNA, 31 bp, Artificial Sequence
  Synthetic nucleic acid sequence
acgttggatggccactctagtactatatctg

SEQ ID NO: 23 - DNA, 30 bp, Artificial Sequence
  Synthetic nucleic acid sequence
acgttggatggggtattcataagctgaaac

SEQ ID NO: 24 - DNA, 30 bp, Artificial Sequence
  Synthetic nucleic acid sequence
acgttggatgccttcaagttcagtggtcag

SEQ ID NO: 25 - DNA, 30 bp, Artificial Sequence
  Synthetic nucleic acid sequence
acgttggatgggtcaatgaagagaacttgg

SEQ ID NO: 26 - DNA, 30 bp, Artificial Sequence
  Synthetic nucleic acid sequence
acgttggatgaatgtttattgtagaaaacc

SEQ ID NO: 27 - DNA, 15 bp, Artificial Sequence
  Synthetic nucleic acid sequence
agctttgccagttcc

SEQ ID NO: 28 - DNA, 18 bp, Artificial Sequence
  Synthetic nucleic acid sequence
aaaagcaaaattgcctga

SEQ ID NO: 29 - DNA, 15 bp, Artificial Sequence
  Synthetic nucleic acid sequence
cacgacgtcacgcag

SEQ ID NO: 30 - DNA, 17 bp, Artificial Sequence
  Synthetic nucleic acid sequence
acctccgctgcaaatac

SEQ ID NO: 31 - DNA, 18 bp, Artificial Sequence
  Synthetic nucleic acid sequence
gagtctggacacgtgggg

SEQ ID NO: 32 - DNA, 19 bp, Artificial Sequence
  Synthetic nucleic acid sequence
tccatctctgtggatctcc

SEQ ID NO: 33 - DNA, 19 bp, Artificial Sequence
  Synthetic nucleic acid sequence
tgctgcaggccccagatga

SEQ ID NO: 34 - DNA, 21 bp, Artificial Sequence
  Synthetic nucleic acid sequence
cacaatttggtgaattatcaa

SEQ ID NO: 35 - DNA, 15 bp, Artificial Sequence
  Synthetic nucleic acid sequence
agctgaaacttctgg

SEQ ID NO: 36 - DNA, 20 bp, Artificial Sequence
  Synthetic nucleic acid sequence
tcaagcttgccaaagtaatc

SEQ ID NO: 37 - DNA, 16 bp, Artificial Sequence
  Synthetic nucleic acid sequence
agctttgccagttcct

SEQ ID NO: 38 - DNA, 17 bp, Artificial Sequence
  Synthetic nucleic acid sequence
agctttgccagttccgt

SEQ ID NO: 39 - DNA, 19 bp, Artificial Sequence
  Synthetic nucleic acid sequence
aaaagcaaaattgcctgat

SEQ ID NO: 40 - DNA, 21 bp, Artificial Sequence
  Synthetic nucleic acid sequence
aaaagcaaaattgcctgaggc

SEQ ID NO: 41 - DNA, 16 bp, Artificial Sequence
  Synthetic nucleic acid sequence
cacgacgtcacgcagc

SEQ ID NO: 42 - DNA, 17 bp, Artificial Sequence
  Synthetic nucleic acid sequence
cacgacgtcacgcagga

SEQ ID NO: 43 - DNA, 18 bp, Artificial Sequence
  Synthetic nucleic acid sequence
acctccgctgcaaataca

SEQ ID NO: 44 - DNA, 19 bp, Artificial Sequence
  Synthetic nucleic acid sequence
acctccgctgcaaatacgt

SEQ ID NO: 45 - DNA, 19 bp, Artificial Sequence
  Synthetic nucleic acid sequence
gagtctggacacgtgggga

SEQ ID NO: 46 - DNA, 20 bp, Artificial Sequence
  Synthetic nucleic acid sequence
gagtctggacacgtggggga

SEQ ID NO: 47 - DNA, 20 bp, Artificial Sequence
  Synthetic nucleic acid sequence
tccatctctgtggatctcca

SEQ ID NO: 48 - DNA, 21 bp, Artificial Sequence
  Synthetic nucleic acid sequence
tccatctctgtggatctccgt

SEQ ID NO: 49 - DNA, 20 bp, Artificial Sequence
  Synthetic nucleic acid sequence
tgctgcaggccccagatgat

SEQ ID NO: 50 - DNA, 21 bp, Artificial Sequence
  Synthetic nucleic acid sequence
tgctgcaggccccagatgagc

SEQ ID NO: 51 - DNA, 22 bp, Artificial Sequence
  Synthetic nucleic acid sequence
cacaatttggtgaattatcaat

SEQ ID NO: 52 - DNA, 23 bp, Artificial Sequence
  Synthetic nucleic acid sequence
cacaatttggtgaattatcaaat

SEQ ID NO: 53 - DNA, 16 bp, Artificial Sequence
  Synthetic nucleic acid sequence
agctgaaacttctggc

SEQ ID NO: 54 - DNA, 17 bp, Artificial Sequence
  Synthetic nucleic acid sequence
agctgaaacttctggga

SEQ ID NO: 55 - DNA, 21 bp, Artificial Sequence
  Synthetic nucleic acid sequence
tcaagcttgccaaagtaatct

SEQ ID NO: 56 - DNA, 23 bp, Artificial Sequence
  Synthetic nucleic acid sequence
tcaagcttgccaaagtaatcgga

(19) United States
(12) Patent Application Publication
(10) Pub. No.: US 2006/0281114 A1
(43) Pub. Date: Dec. 14, 2006

(54) Methods and compositions for assessment of pulmonary function and disorders

(76) Inventor: Robert Peter Young, Parnell (NZ)

Correspondence Address: KNOBBE MARTENS OLSON & BEAR LLP, 2040 MAIN STREET, FOURTEENTH FLOOR, IRVINE, CA, 92614, US

(21) Appl. No.: 11/438,082

(22) Filed: May 19, 2006

(30) Foreign Application Priority Data
May 19, 2005 (NZ) 540202
Jul. 20, 2005 (NZ) 541389

(51) Int. Cl. C12Q 1/68; G01N 33/567; C07H 21/04

(52) U.S. Cl. 435/6; 435/7.2; 536/24.3

(57) ABSTRACT

The present invention provides methods for the assessment of risk of developing occupational chronic obstructive pulmonary disease (OCOPD) in smokers and non-smokers using analysis of genetic polymorphisms. The present invention also relates to the use of genetic polymorphisms in assessing a subject's risk of developing OCOPD. Nucleotide probes and primers, kits, and microarrays suitable for such assessment are also provided.

### CROSS-REFERENCE TO RELATED APPLICATIONS

This application claims priority to: New Zealand Application No. 540202, filed May 19, 2005; and New Zealand Application No. 541389, filed Jul. 20, 2005, both of which are incorporated by reference in their entireties.

### TECHNICAL FIELD

The present invention is concerned with methods for assessment and treatment of pulmonary function and/or disorders, and in particular for assessing risk of developing occupational chronic obstructive pulmonary disease (OCOPD). The present invention is also concerned with the use of genetic polymorphisms in the assessment of a subject's risk of developing OCOPD.

### BACKGROUND OF THE INVENTION

Occupational chronic obstructive pulmonary disease (OCOPD) is a well-recognized and well-studied consequence of chronic exposure to a diverse range or aero-pollutants in the workplace. A recent document published by the American Thoracic Society on the occupational contribution to COPD estimates that 15% of all COPD is work related with annual costs of US$7 billion [1, herein incorporated by reference in its entirety]. OCOPD is ranked the second highest cause of occupationally related death and believed to be on the rise [2, herein incorporated by reference in its entirety].

Both cross sectional and prospective studies have shown that occupational COPD occurs in a range of occupations characterized by chronic exposure to dust and/or other aero-pollutants including organic and inorganic aero-pollutants [reviewed in 3 and 4, each of the foregoing is herein incorporated by reference in its entirety]. These occupations and industries include metallurgy, iron and steel workers, wood processing workers, chemistry and chemical workers, pulp and paper manufacturing, printing industry, farmers, armed forces, flour milling, popcorn manufacturing, coal, gold, silica and rock miners, welders, painters, boat builders, cotton/synthetic textile workers, construction workers, tobacco workers, and ammonia workers. Examples of pollutants associated with OCOPD include heavy metals (including Cadmium and Vanadium), Nitrogen dioxide, Sulphur dioxide, grain dust, endotoxin, solvents and resins.

In two separate studies, it is estimated that around 40 million people in the United States work force are employed in the “at risk” occupations listed above [5, 6, each of the foregoing which is herein incorporated by reference in its entirety].

Studies show that OCOPD results from host factors (including genetic makeup) in combination with exposure dose (for example, concentration and duration). It has been estimated that about 20% of those workers in these occupations can be susceptible to OCOPD.

Importantly, the link between the above occupations and risk of OCOPD is independent of the effects of smoking, ethnicity, and age. In nonsmokers it has been shown that the effect from repeated exposure to the dusts or fumes from the above occupations is equivalent to the effect of smoking in inducing COPD. Moreover, for smokers the combined effect of their smoking and occupational exposure on decline in lung function is greater than either one alone. Therefore, smokers who are also exposed to aero-pollutants at work are at significant risk.

Occupational chronic obstructive pulmonary disease is characterised by insidious inflammation and progressive lung destruction. It becomes clinically evident after exertional breathlessness is noted by affected subjects when 50% or more of lung function has already been irreversibly lost. This loss of lung function is detected clinically by reduced expiratory flow rates (specifically forced expiratory volume in one second or FEV1).

Despite advances in the treatment of airways disease, current therapies do not significantly alter the natural history of OCOPD with progressive loss of lung function causing respiratory failure and death. Although cessation of occupational exposure may be expected to reduce this decline in lung function, it is probable that if this is not achieved at an early stage, the loss is considerable and symptoms of worsening breathlessness likely cannot be averted. Analogous to the discovery of serum cholesterol and its link to coronary artery disease, there is a need to better understand the factors that contribute to OCOPD so that tests that identify at risk workers can be developed and that new treatments can be discovered to reduce the adverse effects of occupational exposure to pollutants.

To date, a number of biomarkers useful in the diagnosis and assessment of propensity towards developing various pulmonary disorders have been identified. These include, for example, single nucleotide polymorphisms including the following: A-82G in the promoter of the gene encoding human macrophage elastase (MMP12); T→C within codon 10 of the gene encoding transforming growth factor beta (TGFβ); C+760G of the gene encoding superoxide dismutase 3 (SOD3); T-1296C within the promoter of the gene encoding tissue inhibitor of metalloproteinase 3 (TIMP3); and polymorphisms in linkage disequilibrium with these polymorphisms, as disclosed in PCT International Application PCT/NZ02/00106 (published as WO 02/099134 and herein incorporated by reference in its entirety).

### SUMMARY OF THE INVENTION

In some aspects, the present invention is primarily based on the finding that certain polymorphisms are found more often in subjects with OCOPD than in control subjects. Analysis of these polymorphisms reveals an association between genotypes and the subject's risk of developing OCOPD.

Thus, according to one aspect there is provided a method of determining a subject's risk of developing occupational chronic obstructive pulmonary disease including analyzing a sample from said subject for the presence or absence of one or more polymorphisms selected from the group consisting of: −765 C/G in the promoter of the gene encoding cyclooxygenase 2 (COX2), Ile 105 Val (A/G) in the gene encoding glutathione S transferase P (GSTP1); 105 C/A in the gene encoding interleukin-18 (IL-18); −133 G/C in the promoter of the gene encoding IL-18; −251 A/T in the gene encoding interleukin-8 (IL-8); Lys 420 Thr (A/C) in the gene encoding Vitamin D binding protein (VDBP); Glu 416 Asp (T/G) in the gene encoding VDBP; exon 3 T/C (R/r) in the gene encoding microsomal epoxide hydrolase (MEH); Arg 312 Gln (AC) in the gene encoding superoxide dismutase 3 (SOD3); 3′ 1237 G/A (T/t) in the gene encoding α1-antitrypsin; α1-antitrypsin (α1AT) S polymorphism; Asp 299 Gly A/G in the gene encoding toll-like receptor 4 (TLR4); Gln27Glu in the gene encoding β2 adrenoreceptor (ADRB2); −518 G/A in the promoter of the gene encoding interleukin-11 (IL-11); −1055 (C/T) in the promoter of the gene encoding interleukin-13 (IL-13); −675 4G/5G in the promoter of the gene encoding plasminogen activator inhibitor 1 (PAI-1); 298 Asp/Glu (T/G) in the gene encoding nitric oxide synthase 3 (NOS3); −1607 1G/2G in the gene encoding matrix metalloproteinase 1 (MMP1); wherein the presence or absence of one or more of said polymorphisms is indicative of the subject's risk of developing occupational chronic obstructive pulmonary disease.

The one or more polymorphisms can be detected directly or by detection of one or more polymorphisms which are in linkage disequilibrium with said one or more polymorphisms.

Linkage disequilibrium (LD) is a phenomenon in genetics whereby two or more mutations or polymorphisms are in such close genetic proximity that they are co-inherited. This means that in genotyping, detection of one polymorphism as present implies the presence of the other. (Reich D E et al; Linkage disequilibrium in the human genome, Nature 2001, 411:199-204, herein incorporated by reference in its entirety.)

In some aspects, the presence of one or more polymorphisms selected from the group consisting of: −765 CC or CG in the promoter of the gene encoding COX2; −251 AA genotype in the promoter of the gene encoding IL-8; Lys 420 Thr AA genotype in the gene encoding VDBP; Glu 416 Asp TT or TG genotype in the gene encoding VDBP; exon 3 T/C RR genotype in the gene encoding MEH; Arg 312 Gln AG or GG genotype in the gene encoding SOD3; MS or SS genotype in the gene encoding α1AT; Asp 299 Gly AG or GG genotype in the gene encoding TLR4; Gln 27 Glu CC genotype in the gene encoding ADRB2; −5.18 AA genotype in the gene encoding IL-11; and Asp 298 Glu TT genotype in the gene encoding NOS3; is indicative of a reduced risk of developing OCOPD.

In some aspects, the presence of one or more polymorphisms selected from the group consisting of: −765 GG in the promoter of the gene encoding COX2; Ile 105 Val GG in the gene encoding GSTP1; 105 AA in the gene encoding IL-18; −133 CC in the promoter of the gene encoding IL-18; Lys 420 Thr CC in the gene encoding VDBP; Glu 416 Asp GG in the gene encoding VDBP; Arg 312 Gln AA in the gene encoding SOD3; 3′ 1237 G/A (T/t) in the gene encoding α1-antitrypsin; −1055 TT in the promoter of the gene encoding IL-13; −675 5G5G in the promoter of the gene encoding PAI-1; and −1607 2G2G in the gene encoding matrix metalloproteinase 1 (MMP1); is indicative of an increased risk of developing OCOPD.

The polymorphisms can be analyzed alone or, more preferably, in any combination of two or more.

The methods are particularly useful in subjects chronically exposed to aero-pollutants, preferably subjects whose occupation or former occupation is or was associated with exposure to aero-pollutants. Said methods are also particularly useful in such subjects who are or were smokers.

It will be appreciated that the methods of the invention identify two categories of polymorphisms—namely those associated with a reduced risk of developing OCOPD (which can be termed “protective polymorphisms”) and those associated with an increased risk of developing OCOPD (which can be termed “susceptibility polymorphisms”).

Therefore, the present invention further provides a method of assessing a subject's risk of developing occupational chronic obstructive pulmonary disease (OCOPD), said method including: determining the presence or absence of at least one protective polymorphism associated with a reduced risk of developing OCOPD; and in the absence of at least one protective polymorphism, determining the presence or absence of at least one susceptibility polymorphism associated with an increased risk of developing OCOPD. The presence of one or more of said protective polymorphisms is indicative of a reduced risk of developing OCOPD, and the absence of at least one protective polymorphism in combination with the presence of at least one susceptibility polymorphism is indicative of an increased risk of developing OCOPD.

In some embodiments, said at least one protective polymorphism is selected from the group consisting of: the −765 CC or CG genotype in the promoter of the gene encoding COX2; the −251 AA genotype in the promoter of the gene encoding IL-8; the Lys 420 Thr AA genotype in the gene encoding VDBP; the Glu 416 Asp TT or TG genotype in the gene encoding VDBP; the exon 3 T/C RR genotype in the gene encoding MEH; the Arg 312 Gln AG or GG genotype in the gene encoding SOD3; the MS or SS genotype in the gene encoding α1AT; the Asp 299 Gly AG genotype in the gene encoding TLR4; the Gln 27 Glu CC genotype in the gene encoding ADRB2; the −518 AA genotype in the gene encoding IL-11; and the Asp 298 Glu TT genotype in the gene encoding NOS3.

Optionally, said method includes the additional step of determining the presence or absence of at least one further protective polymorphism selected from the group consisting of: the +760 GG or +760 CG genotype within the gene encoding SOD3; the −1296 TT genotype within the promoter of the gene encoding TIMP3; the CC genotype (homozygous P allele) within codon 10 of the gene encoding TGFβ; and 2G2G within the promoter of the gene encoding MMP1.

In some aspects, the at least one susceptibility polymorphism is a genotype selected from the group consisting of: the −765 GG genotype in the promoter of the gene COX2; the Ile 105 Val GG genotype in the gene encoding GSTP1; the 105 AA genotype in the gene encoding IL-18; −133 CC genotype in the promoter of the gene encoding IL-18; the Lys 420 Thr CC genotype in the gene encoding VDBP; the Glu 416 Asp GG genotype in the gene encoding VDBP; the Arg 312 Gln AA genotype in the gene encoding SOD3; the −1055 TT genotype in the promoter of the gene encoding IL-13; the −675 5G5G genotype in the promoter of the gene encoding PAI-1; the 1237 Tt or tt genotype in the gene encoding α1AT; and the −1607 2G2G genotype in the gene encoding MMP1.

Optionally, said method includes the step of determining the presence or absence of at least one further susceptibility polymorphism selected from the group consisting of: the −82 AA genotype within the promoter of the gene encoding MMP12; and the −1562 CT or −1562 TT genotype within the promoter of the gene encoding MMP9.

In some embodiments, the presence of two or more protective polymorphisms is indicative of a reduced risk of developing OCOPD.

In some embodiments, the presence of two or more susceptibility polymorphisms is indicative of an increased risk of developing OCOPD.

In some embodiments, the presence of two or more protective polymorphims irrespective of the presence of one or more susceptibility polymorphisms is indicative of reduced risk of developing OCOPD.

In another aspect, the invention provides a method of determining a subject's risk of developing OCOPD, said method including obtaining the result of one or more genetic tests of a sample from said subject, and analyzing the result for the presence or absence of one or more polymorphisms selected from the group consisting of: −765 C/G in the promoter of the gene encoding cyclooxygenase 2 (COX2); Ile 105 Val (A/G) in the gene encoding glutathione S transferase P (GSTP1); 105 C/A in the gene encoding interleukin-18 (IL-18); −133 G/C in the promoter of the gene encoding IL-18; −251 A/T in the gene encoding interleukin-8 (IL-8); Lys 420 Thr (A/C) in the gene encoding Vitamin D binding protein (VDBP); Glu 416 Asp (T/G) in the gene encoding VDBP; exon 3 T/C (R/r) in the gene encoding microsomal epoxide hydrolase (MEH); Arg 312 Gln (AC) in the gene encoding superoxide dismutase 3 (SOD3); 3′ 1237 G/A (T/t) in the gene encoding α1-antitrypsin; α1-antitrypsin (α1AT) S polymorphism; Asp 299 Gly A/G in the gene encoding toll-like receptor 4 (TLR4); Gln27Glu in the gene encoding β2 adrenoreceptor (ADRB2); −518 G/A in the promoter of the gene encoding interleukin-11 (IL-11); −1055 (C/T) in the promoter of the gene encoding interleukin-13 (IL-13); −675 4G/5G in the promoter of the gene encoding plasminogen activator inhibitor 1 (PAI-1); 298 Asp/Glu (T/G) in the gene encoding nitric oxide synthase 3 (NOS3); −1607 1G/2G in the gene encoding matrix metalloproteinase 1 (MMP1); and one or more polymorphisms which are in linkage disequilibrium with any one or more of these polymorphisms, wherein a result indicating the presence or absence of one or more of said polymorphisms is indicative of the subject's risk of developing OCOPD.

In a further aspect the invention provides a method of determining a subject's risk of developing occupational chronic obstructive pulmonary disease (OCOPD), said method including determining the presence or absence of the −765 C allele in the promoter of the gene encoding COX2 and/or the S allele in the gene encoding α1AT, wherein the presence of any one or more of said alleles is indicative of a reduced risk of developing OCOPD.

In a further aspect the invention provides a method of determining a subject's risk of developing occupational chronic obstructive pulmonary disease (OCOPD), said method including determining the presence or absence of the −765 CC or CG genotype in the promoter of the gene encoding COX2 and/or the MS genotype in the gene encoding 1-antitrypsin, wherein the presence of any one or more of said genotypes is indicative of a reduced risk of developing OCOPD.

In a further aspect there is provided a method of determining a subject's risk of developing occupational chronic obstructive pulmonary disease (OCOPD) including the analysis of two or more polymorphisms selected from the group consisting of: −765 C/G in the promoter of the gene encoding cyclooxygenase 2 (COX2); Ile 105 Val (A/G) in the gene encoding glutathione S transferase P (GSTP1); 105 C/A in the gene encoding interleukin-18 (IL-18); −133 G/C in the promoter of the gene encoding IL-18; −251 A/T in the gene encoding interleukin-8 (IL-8); Lys 420 Thr (A/C) in the gene encoding Vitamin D binding protein (VDBP); Glu 416 Asp (T/G) in the gene encoding VDBP; exon 3 T/C (R/r) in the gene encoding microsomal epoxide hydrolase (MEH); Arg 312 Gln (AC) in the gene encoding superoxide dismutase 3 (SOD3); α1-antitrypsin (α1AT) S polymorphism; Asp 299 Gly A/G in the gene encoding toll-like receptor 4 (TLR4); Gln27Glu in the gene encoding β2 adrenoreceptor (ADRB2); −518 G/A in the promoter of the gene encoding interleukin-11 (IL-11); −1055 (C/T) in the promoter of the gene encoding interleukin-13 (IL-13); −675 4G/5G in the promoter of the gene encoding plasminogen activator inhibitor 1 (PAI-1); 298 Asp/Glu (T/G) in the gene encoding nitric oxide synthase 3 (NOS3); 3′ 1237 G/A (T/t) in the gene encoding α/AT; and −1607 1G/2G in the gene encoding MMP1.

In various embodiments, any one or more of the above methods includes the step of analyzing the amino acid present at a position mapping to codon 420 of the gene encoding VDBP.

In some embodiments, the presence of threonine at said position is indicative of an increased risk of developing OCOPD.

In some embodiments, the presence of lysine at said position is indicative of reduced risk of developing OCOPD.

In various embodiments, any one or more of the above methods includes the step of analyzing the amino acid present at a position mapping to codon 416 of the gene encoding VDBP.

In various embodiments, any one or more of the above methods includes the step of analyzing the amino acid present at a position mapping to codon 312 of the gene encoding SOD3.

In various embodiments, any one or more of the above methods includes the step of analyzing the amino acid present at a position mapping to codon 299 of the gene encoding TLR4.

In various embodiments, any one or more of the above methods includes the step of analyzing the amino acid present at a position mapping to codon 27 of the gene encoding ADRB2.

In various embodiments, any one or more of the above methods includes the step of analyzing the amino acid present at a position mapping to codon 298 of the gene encoding NOS3.

In some embodiments, the presence of glutamate at said position is indicative of an increased risk of developing OCOPD.

In some embodiments, the presence of asparagine at said position is indicative of reduced risk of developing OCOPD.

In a preferred form of the invention the methods as described herein are performed in conjunction with an analysis of one or more risk factors, including one or more epidemiological risk factors, associated with a risk of developing occupational chronic obstructive pulmonary disease (OCOPD). Such epidemiological risk factors include but are not limited to smoking or exposure to tobacco smoke, age, sex, and familial history of OCOPD.

In a further aspect, the invention provides for the use of at least one polymorphism in the assessment of a subject's risk of developing OCOPD, wherein said at least one polymorphism is selected from the group consisting of: −765 C/G in the promoter of the gene encoding cyclooxygenase 2 (COX2); Ile 105 Val (A/G) in the gene encoding glutathione S transferase P (GSTP1); 105 C/A in the gene encoding interleukin-18 (IL-18); −133 G/C in the promoter of the gene encoding IL-18; −251 A/T in the gene encoding interleukin-8 (IL-8); Lys 420 Thr (A/C) in the gene encoding Vitamin D binding protein (VDBP); Glu 416 Asp (T/G) in the gene encoding VDBP; exon 3 T/C (R/r) in the gene encoding microsomal epoxide hydrolase (MEH); Arg 312 Gln (AC) in the gene encoding superoxide dismutase 3 (SOD3); 3′ 1237 G/A (T/t) in the gene encoding α1-antitrypsin; α1-antitrypsin (α1AT) S polymorphism; Asp 299 Gly A/G in the gene encoding toll-like receptor 4 (TLR4); Gln27Glu in the gene encoding β2 adrenoreceptor (ADRB2); −518 G/A in the promoter of the gene encoding interleukin-11 (IL-11); −1055 (C/T) in the promoter of the gene encoding interleukin-13 (IL-13); −675 4G/5G in the promoter of the gene encoding plasminogen activator inhibitor 1 (PAI-1); 298 Asp/Glu (T/G) in the gene encoding nitric oxide synthase 3 (NOS3); −1607 1G/2G in the gene encoding matrix metalloproteinase 1 (MMP1); and one or more polymorphisms in linkage disequilibrium with any one of said polymorphisms.

In another aspect the invention provides a set of nucleotide probes and/or primers for use in the preferred methods of the invention herein described. Preferably, the nucleotide probes and/or primers are those which span, or are able to be used to span, the polymorphic regions of the genes. Also provided are one or more nucleotide probes and/or primers comprising the sequence of any one of the probes and/or primers herein described, including any one comprising the sequence of any one of SEQ. ID. NO.1 to SEQ. ID. NO.56, more preferably any one of SEQ. ID. NO. 7 to SEQ. ID. NO.56.

In yet a further aspect, the invention provides a nucleic acid microarray for use in the methods of the invention, which microarray includes a substrate presenting nucleic acid sequences capable of hybridizing to nucleic acid sequences which encode one or more of the susceptibility or protective polymorphisms described herein or sequences complimentary thereto.

In another aspect, the invention provides an antibody microarray for use in the methods of the invention, which microarray includes a substrate presenting antibodies capable of binding to a product of expression of a gene the expression of which is upregulated or downregulated when associated with a susceptibility or protective polymorphism as described herein.

In a further aspect the present invention provides a method treating a subject having an increased risk of developing OCOPD including the step of replicating, genotypically or phenotypically, the presence and/or functional effect of a protective polymorphism in said subject.

In yet a further aspect, the present invention provides a method of treating a subject having an increased risk of developing OCOPD said subject having a detectable susceptibility polymorphism which either upregulates or downregulates expression of a gene such that the physiologically active concentration of the expressed gene product is outside a range which is normal for the age and sex of the subject, said method including the step of restoring the physiologically active concentration of said product of gene expression to be within a range which is normal for the age and sex of the subject.

In yet a further aspect the present invention provides a method of treating a subject having an increased risk of developing OCOPD and for whom the presence of the GG genotype at the −765 C/G polymorphism present in the promoter of the gene encoding COX2 has been determined, said method including administering to said subject an agent capable of reducing COX2 activity in said subject.

In one embodiment, said agent is a COX2 inhibitor or a nonsteroidal anti-inflammatory drug (NSAID), preferably said COX2 inhibitor is selected from the group consisting of Celebrex (Celecoxib), Bextra (Valdecoxib), and Vioxx (Rofecoxib).

In a further aspect the present invention provides a method of treating a subject having an increased risk of developing OCOPD and for whom the presence of the AA genotype at the 105 C/A polymorphism in the gene encoding IL-18 has been determined, said method including administering to said subject an agent capable of augmenting IL-18 activity in said subject.

In yet a further aspect the present invention provides a method of treating a subject having an increased risk of developing OCOPD and for whom the presence of the CC genotype at the −133 G/C polymorphism in the promoter of the gene encoding IL-18 has been determined, said method including administering to said subject an agent capable of augmenting IL-18 activity in said subject.

In still a further aspect the present invention provides a method of treating a subject having an increased risk of developing OCOPD and for whom the presence of the 5G5G genotype at the −675 4G/5G polymorphism in the promoter of the gene encoding PAI-1 has been determined, said method including administering to said subject an agent capable of augmenting PAI-1 activity in said subject.

In yet a further aspect, the present invention provides a method for screening for compounds that modulate the expression and/or activity of a gene, the expression of which is upregulated or downregulated when associated with a susceptibility or protective polymorphism, said method including the steps of: contacting a candidate compound with a cell including a susceptibility or protective polymorphism which has been determined to be associated with the upregulation or downregulation of expression of a gene; and measuring the expression of said gene following contact with said candidate compound, wherein a change in the level of expression after the contacting step as compared to before the contacting step is indicative of the ability of the compound to modulate the expression and/or activity of said gene.

In some embodiments, preferably, said cell is a human lung cell which has been pre-screened to confirm the presence of said polymorphism.

In some embodiments, preferably, said cell includes a susceptibility polymorphism associated with upregulation of expression of said gene and said screening is for candidate compounds which downregulate expression of said gene.

Alternatively, said cell contains a susceptibility polymorphism associated with downregulation of expression of said gene and said screening is for candidate compounds which upregulate expression of said gene.

In another embodiment, said cell contains a protective polymorphism associated with upregulation of expression of said gene and said screening is for candidate compounds which further upregulate expression of said gene.

Alternatively, said cell includes a protective polymorphism associated with downregulation of expression of said gene and said screening is for candidate compounds which further downregulate expression of said gene.

In another aspect, the present invention provides a method for screening for compounds that modulate the expression and/or activity of a gene, the expression of which is upregulated or downregulated when associated with a susceptibility or protective polymorphism, said method including the steps of contacting a candidate compound with a cell including a gene, the expression of which is upregulated or downregulated when associated with a susceptibility or protective polymorphism but which in said cell the expression of which is neither upregulated nor downregulated; and measuring the expression of said gene following contact with said candidate compound, wherein a change in the level of expression after the contacting step as compared to before the contacting step is indicative of the ability of the compound to modulate the expression and/or activity of said gene.

In some embodiments, preferably, said cell is a human lung cell which has been pre-screened to confirm the presence, and baseline level of expression, of said gene.

In some embodiments, preferably, expression of the gene is down-regulated when associated with a susceptibility polymorphism once said screening is for candidate compounds which in said cell, upregulate expression of said gene.

Alternatively, expression of the gene is upregulated when associated with a susceptibility polymorphism and said screening is for candidate compounds which, in said cell, downregulate expression of said gene.

In another embodiment, expression of the gene is upregulated when associated with a protective polymorphism and said screening is for compounds which, in said cell, upregulate expression of said gene.

Alternatively, expression of the gene is downregulated when associated with a protective polymorphism and said screening is for compounds which, in said cell, downregulate expression of said gene.

In yet a further aspect, the present invention provides a method of assessing the likely responsiveness of a subject having an increased risk of developing OCOPD to a prophylactic or therapeutic treatment, which treatment involves restoring the physiologically active concentration of a product of gene expression to be within a range which is normal for the age and sex of the subject, which method includes detecting in said subject the presence or absence of a susceptibility polymorphism which when present either upregulates or downregulates expression of said gene such that the physiological active concentration of the expressed gene product is outside said normal range, wherein the detection of the presence of said polymorphism is indicative of the subject likely responding to said treatment.

In a further aspect, the present invention provides a kit for assessing a subject's risk of developing OCOPD, said kit including a means of analyzing a sample from said subject for the presence or absence of one or more polymorphisms disclosed herein.

### BRIEF DESCRIPTION OF THE DRAWINGS

FIG. 1 depicts a graph showing the percentage of people with OCOPD plotted against the number of protective genetic variants.

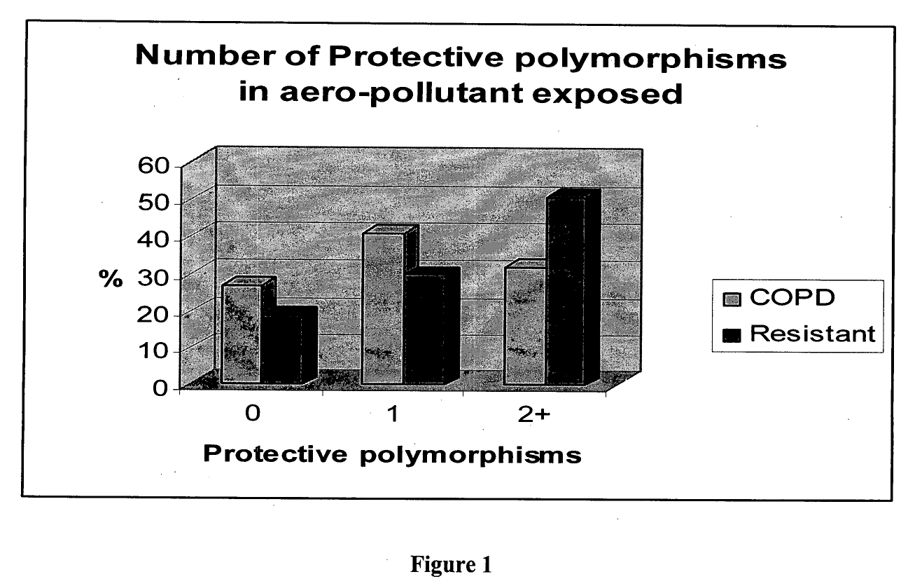

FIG. 2 depicts a graph showing the percentage of people with OCOPD plotted against the number of susceptibility genetic variants.

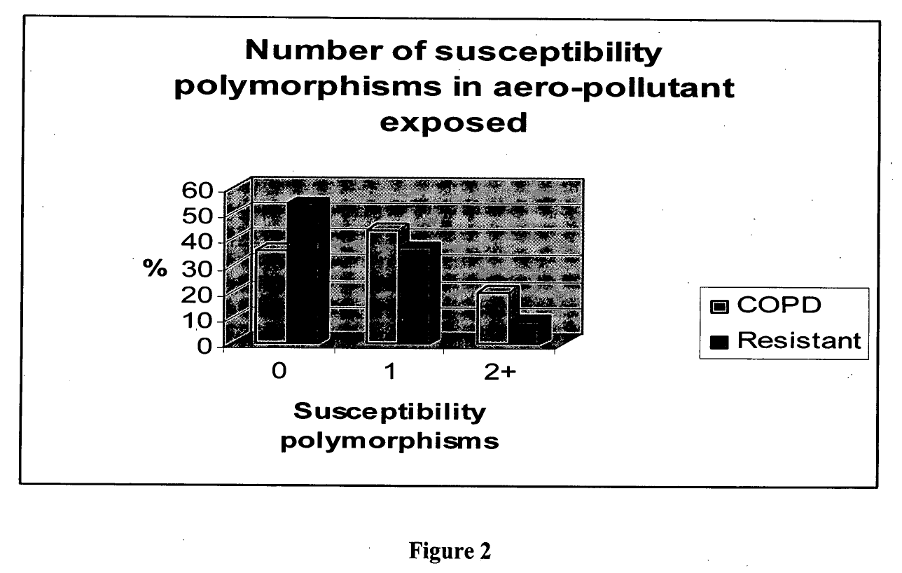

### DETAILED DESCRIPTION OF THE PREFERRED EMBODIMENT

Additional biomarkers that can be used to assess a subject's risk of developing pulmonary disorders such as occupational chronic obstructive pulmonary disease (OCOPD), or risk of developing OCOPD-related impaired lung function, particularly if the subject is a smoker, can be desirable. In some aspects, it is primarily to such biomarkers and their use in methods to assess risk of developing such disorders that the present invention is directed.

Using case-control studies the frequencies of several genetic variants (polymorphisms) of candidate genes in smokers who have developed OCOPD, smokers who appear resistant to OCOPD, and blood donor controls have been compared. The majority of these candidate genes have confirmed (or likely) functional effects on gene expression or protein function. Specifically the frequencies of polymorphisms between blood donor controls, resistant smokers and those with OCOPD (subdivided into those with early onset and those with normal onset) have been compared. The present invention demonstrates that there are both protective and susceptibility polymorphisms present in selected candidate genes of the patients tested.

Importantly, a number of the polymorphisms found to be discriminatory of the risk of developing OCOPD were found to not be discriminatory of the risk of developing COPD, emphysema, or both COPD and emphysema (for example, COPD and emphysema associated with smoking), as discussed in pending Application NZ 539934. For example, the SOD3 Arg 312 Gln polymorphism, the TLR4 Asp 299 Gly A/G polymorphism, the IL-8-251 A/T polymorphism, the IL-11 −518 G/A polymorphism, and the MEH Exon 3 T/C (r/R) polymorphism are each discriminatory for OCOPD, but not for COPD. Conversely, a number of polymorphisms determined to be discriminatory of the risk of developing COPD and/or emphysema (as discussed in NZ 539934) are not discriminatory of the risk of developing OCOPD. For example, the interferon-γ 874 A/T polymorphism and the interleukin-13 Arg 130 Gln polymorphism are each discriminatory for COPD and/or emphysema, but not for OCOPD. Thus, without wishing to be bound by any theory, it appears the discriminatory value of a given polymorphism for determining risk of developing a particular disease or disorder cannot be readily predicted on the basis of discriminatory value in another, albeit related, disease or disorder. A specific association between any given polymorphism and the relevant disease or disorder is necessary.

The present invention identifies both protective and susceptibility genotypes for OCOPD derived from selected candidate gene polymorphisms. Specifically, 11 susceptibility polymorphisms and 11 protective polymorphisms are identified. These are as follows in Table 1A:

TABLE 1A

| Gene | Polymorphism | Role |
|---|---|---|
| Cyclooxygenase 2 (COX2) | COX2-765 G/C | CC/CG protective |
| | | GG susceptibility |
| β2-adrenoreceptor (ADRB2) | ADRB2 Gln 27 Glu | CC protective |
| Interleukin-18 (IL-18) | IL-18-133 C/G | CC susceptibility |
| Interleukin-18 (IL-18) | IL-18 105 A/C | AA susceptibility |
| Plasminogen activator inhibitor 1 (PAI-1) | PAI-1-675 4G/5G | 5G5G susceptibility |
| Nitric Oxide synthase 3 (NOS3) | NOS3 298 Asp/Glu | TT protective |
| Vitamin D Binding Protein (VDBP) | VDBP Lys 420 Thr | AA protective |
| | | CC susceptibility |
| Vitamin D Binding Protein (VDBP) | VDBP Glu 416 Asp | TT/TG protective |
| | | GG susceptibility |
| Glutathione S Transferase (GSTP1) | GSTP1 Ile 105 Val | GG susceptibility |
| Superoxide dismutase 3 (SOD3) | SOD3 Arg 312 Gln | AG/GG protective |
| | | AA susceptibility |
| α1-antitrypsin (α1AT) | α/AT 3′1237 G/A (T/A) | Tt/tt susceptibility |
| α1-antitrypsin (α1AT) | α1AT S allele | MS protective |
| Toll-like receptor 4 (TLR4) | TLR4 Asp 299 Gly A/G | AG/GG protective |
| Interleukin-8 (IL-8) | IL-8-251 A/T | AA protective |
| Interleukin 11 (IL-11) | IL-11-518 G/A | AA protective |
| Microsomal epoxide hydrolase (MEH) | MEH Exon 3 T/C (r/R) | RR protective |
| Interleukin 13 (IL-13) | IL-13-1055 C/T | TT susceptibility |
| Matrix metalloproteinase (MMP1) | MMP1-1607 1G/2G | 2G2G susceptibility |

A susceptibility genetic polymorphism is one which, when present, is indicative of an increased risk of developing OCOPD. In contrast, a protective genetic polymorphism is one which, when present, is indicative of a reduced risk of developing OCOPD.

As used herein, the phrase “risk of developing OCOPD” means the likelihood that a subject to whom the risk applies will develop OCOPD and includes predisposition to, and potential onset of the disease. Accordingly, the phrase “increased risk of developing OCOPD” means that a subject having such an increased risk possesses an hereditary inclination or tendency to develop OCOPD. This does not mean that such a person will actually develop OCOPD at any time, merely that he or she has a greater likelihood of developing OCOPD compared to the general population of individuals that either does not possess a polymorphism associated with increased OCOPD risk, or does possess a polymorphism associated with decreased OCOPD risk. Subjects with an increased risk of developing OCOPD include those with a predisposition to OCOPD, emphysema such as a tendency or prediliction regardless of their lung function at the time of assessment, for example, a subject who is genetically inclined to OCOPD but who has normal lung function, those at potential risk, including subjects with a tendency to mildly reduced lung function who are likely to go on to suffer OCOPD if they keep smoking, and subjects with potential onset of OCOPD who have a tendency to poor lung function on spirometry etc., consistent with OCOPD at the time of assessment.

Similarly, the phrase “decreased risk of developing OCOPD” means that a subject having such a decreased risk possesses a hereditary disinclination or reduced tendency to develop OCOPD. This does not mean that such a person will not develop OCOPD at any time, merely that he or she has a decreased likelihood of developing OCOPD compared to the general population of individuals that either does possess one or more polymorphisms associated with increased OCOPD risk, or does not possess a polymorphism associated with decreased OCOPD risk.

It will be understood that in the context of the present invention the term “polymorphism” means the occurrence together in the same population at a rate greater than that attributable to random mutation (usually greater than 1%) of two or more alternate forms (such as alleles or genetic markers) of a chromosomal locus that differ in nucleotide sequence or have variable numbers of repeated nucleotide units. See world wide web “dot” ornl “dot” gov/sci/techresources/Human_Genome/publicat/97pr/09gloss “dot” html#p. Accordingly, the term “polymorphisms” is used herein contemplates genetic variations, including single nucleotide substitutions, insertions and deletions of nucleotides, repetitive sequences (such as microsatellites), and the total or partial absence of genes (eg. null mutations). As used herein, the term “polymorphisms” also includes genotypes and haplotypes. A genotype is the genetic composition at a specific locus or set of loci. A haplotype is a set of closely linked genetic markers present on one chromosome which are not easily separable by recombination, tend to be inherited together, and can be in linkage disequilibrium. A haplotype can be identified by patterns of polymorphisms such as SNPs. Similarly, in some embodiments, the term “single nucleotide polymorphism” or “SNP” in the context of the present invention includes single base nucleotide subsitutions and short deletion and insertion polymorphisms. In other embodiments, the term SNP includes only single nucleotide substitutions, deletions and insertions.

A reduced or increased risk of a subject developing OCOPD can be diagnosed by analyzing a sample from said subject for the presence of a polymorphism selected from the group consisting of:
−765 C/G in the promoter of the gene encoding cyclooxygenase 2 (COX2);
Ile 105 Val (A/G) in the gene encoding glutathione S transferase P (GSTP1);
105 C/A in the gene encoding interleukin-18 (IL-18);
−133 G/C in the promoter of the gene encoding IL-18;
−251 A/T in the gene encoding interleukin-8 (IL-8);
Lys 420 Thr (A/C) in the gene encoding Vitamin D binding protein (VDBP);
Glu 416 Asp (T/G) in the gene encoding VDBP;
exon 3 T/C (R/r) in the gene encoding microsomal epoxide hydrolase (MEH);
Arg 312 Gln (AC) in the gene encoding superoxide dismutase 3 (SOD3);
3′ 1237 G/A (T/t) in the gene encoding α1-antitrypsin;
α1-antitrypsin (α1AT) S polymorphism;
Asp 299 Gly A/G in the gene encoding toll-like receptor 4 (TLR4);
Gln 27 Glu in the gene encoding β2 adrenoreceptor (ADRB2);
−518 G/A in the promoter of the gene encoding interleukin-11 (IL-11);
−1055 (C/T) in the promoter of the gene encoding interleukin-13 (IL-13);
−675 4G/5G in the promoter of the gene encoding plasminogen activator inhibitor 1 (PAI-1);
298 Asp/Glu (T/G) in the gene encoding nitric oxide synthase 3 (NOS3);
−1607 1G/2G in the gene encoding matrix metalloproteinase 1 (MMP1);
or one or more polymorphisms which are in linkage disequilibrium with any one or more of the above group.

These polymorphisms can also be analyzed in combinations of two or more, or in combination with other polymorphisms indicative of a subject's risk of developing OCOPD inclusive of the remaining polymorphisms listed above.

Expressly contemplated are combinations of the above polymorphisms with polymorphisms as described in PCT International application PCT/NZ02/00106, published as WO 02/099134 (herein incorporated by reference in its entirety).

Assays which involve combinations of polymorphisms, including those amenable to high throughput, such as those utilizing microarrays, are preferred.

Statistical analyses, particularly of the combined effects of these polymorphisms, show that the genetic analyses of the present invention can be used to determine the risk quotient of any smoker and in particular to identify smokers at greater risk of developing OCOPD. Such combined analysis can be of combinations of susceptibility polymorphisms only, of protective polymorphisms only, or of combinations of both. Analysis can also be step-wise, with analysis of the presence or absence of protective polymorphisms occurring first and then with analysis of susceptibility polymorphisms proceeding only where no protective polymorphisms are present.

Thus, through systematic analysis of the frequency of these polymorphisms in well defined groups of smokers and non-smokers, as described herein, it is possible to implicate certain proteins in the development of OCOPD and improve the ability to identify which smokers are at increased risk of developing OCOPD-related impaired lung function and OCOPD for predictive purposes.

The present results show for the first time that the minority of smokers who develop OCOPD do so because they have one or more of the susceptibility polymorphisms and few or none of the protective polymorphisms defined herein. It is thought that the presence of one or more susceptible polymorphisms, together with the damaging irritant and oxidant effects of smoking, combine to make this group of smokers highly susceptible to developing OCOPD. Additional risk factors, such as familial history, age, weight, pack years, etc., will also have an impact on the risk profile of a subject, and can be assessed in combination with the genetic analyses described herein.

The one or more polymorphisms can be detected directly or by detection of one or more polymorphisms which are in linkage disequilibrium with said one or more polymorphisms. As discussed above, linkage disequilibrium is a phenomenon in genetics whereby two or more mutations or polymorphisms are in such close genetic proximity that they are co-inherited. This means that in genotyping, detection of one polymorphism as present implies the presence of the other. (Reich D E et al; Linkage disequilibrium in the human genome, Nature 2001, 411:199-204.)

Examples of polymorphisms described herein that have been reported to be in linkage disequilibrium are presented herein, and include the Interleukin-18 −133 C/G and 105 A/C polymorphisms, and the Vitamin D binding protein Glu 416 Asp and Lys 420 Thr polymorphisms, as shown below in Table 11B.

TABLE 1B

| | | | Alleles in | LD between | Phenotype in |
|---|---|---|---|---|---|
| Gene | SNPs | rs numbers | LD | alleles | COPD |
| Interleukin-18 | IL18-133 C/G | rs360721 | C allele | Strong LD | CC susceptibility |
| | IL18 105 A/C | rs549908 | A allele | | AA susceptibility |
| VDBP | VDBP Lys 420 Thr | Rs4588 | A allele | Strong LD | AA protective |
| | VDBP Glu 416 | Rs7041 | T allele | | TT/TG protective |
| | Asp | | | | |

It will be apparent that polymorphisms in linkage disequilibrium with one or more other polymorphism associated with increased or decreased risk of developing OCOPD will also provide utility as biomarkers for risk of developing OCOPD. The data presented herein shows that the frequency for SNPs in linkage disequilibrium is very similar. Accordingly, these genetically linked SNPs can be utilized in combined polymorphism analyses to derive a level of risk comparable to that calculated from the original SNP.

It will therefore be apparent that one or more polymorphisms in linkage disequilibrium with the polymorphisms specified herein can be identified, for example, using public data bases. Examples of such polymorphisms reported to be in linkage disequilibrium with the polymorphisms specified herein are presented herein in Table 21.

It will also be apparent that frequently a variety of nomenclatures may exist for any given polymorphism. For example, the polymorphism referred to herein as Arg 312 Gln in the gene encoding SOD3 is believed to have been referred to variously as Arg 213 Gly, +760 G/C, and Arg 231 Gly (rs1799895). When referring to a susceptibility or protective polymorphism as herein described, such alternative nomenclatures are also contemplated by the present invention.

The methods of the invention are primarily directed to the detection and identification of the above polymorphisms associated with OCOPD, which are all single nucleotide polymorphisms. In general terms, a single nucleotide polymorphism (SNP) is a single base change or point mutation resulting in genetic variation between individuals. SNPs occur in the human genome approximately once every 100 to 300 bases, and can occur in coding or non-coding regions. Due to the redundancy of the genetic code, a SNP in the coding region may or may not change the amino acid sequence of a protein product. A SNP in a non-coding region can, for example, alter gene expression by, for example, modifying control regions such as promoters, transcription factor binding sites, processing sites, ribosomal binding sites, and affect gene transcription, processing, and translation.

SNPs can facilitate large-scale association genetics studies, and there has recently been great interest in SNP discovery and detection. SNPs show great promise as markers for a number of phenotypic traits (including latent traits), such as for example, disease propensity and severity, wellness propensity, and drug responsiveness including, for example, susceptibility to adverse drug reactions. Knowledge of the association of a particular SNP with a phenotypic trait, coupled with the knowledge of whether an individual has said particular SNP, can enable the targeting of diagnostic, preventative and therapeutic applications to allow better disease management, to enhance understanding of disease states and to ultimately facilitate the discovery of more effective treatments, such as personalised treatment regimens.

Indeed, a number of databases have been constructed of known SNPs, and for some such SNPs, the biological effect associated with a SNP. For example, the NCBI SNP database “dbSNP” is incorporated into NCBI's Entrez system and can be queried using the same approach as the other Entrez databases such as PubMed and GenBank. This database has records for over 1.5 million SNPs mapped onto the human genome sequence. Each dbSNP entry includes the sequence context of the polymorphism (i.e., the surrounding sequence), the occurrence frequency of the polymorphism (by population or individual), and the experimental method(s), protocols, and conditions used to assay the variation, and can include information associating a SNP with a particular phenotypic trait.

At least in part because of the potential impact on health and wellness, there has been and continues to be a great deal of effort to develop methods that reliably and rapidly identify SNPs. This is no trivial task, at least in part because of the complexity of human genomic DNA, with a haploid genome of 3×10⁹ base pairs, and the associated sensitivity and discriminatory requirements.

Genotyping approaches to detect SNPs well-known in the art include DNA sequencing, methods that require allele specific hybridization of primers or probes, allele specific incorporation of nucleotides to primers bound close to or adjacent to the polymorphisms (often referred to as “single base extension”, or “minisequencing”), allele-specific ligation (joining) of oligonucleotides (ligation chain reaction or ligation padlock probes), allele-specific cleavage of oligonucleotides or PCR products by restriction enzymes (restriction fragment length polymorphisms analysis or RFLP) or chemical or other agents, resolution of allele-dependent differences in electrophoretic or chromatographic mobilities, by structure specific enzymes including invasive structure specific enzymes, or mass spectrometry. Analysis of amino acid variation is also possible where the SNP lies in a coding region and results in an amino acid change.

DNA sequencing allows the direct determination and identification of SNPs. The benefits in specificity and accuracy are generally outweighed for screening purposes by the difficulties inherent in whole genome, or even targeted subgenome, sequencing.

Mini-sequencing involves allowing a primer to hybridize to the DNA sequence adjacent to the SNP site on the test sample under investigation. The primer is extended by one nucleotide using all four differentially tagged fluorescent dideoxynucleotides (A,C,G, or T), and a DNA polymerase. Only one of the four nucleotides (homozygous case) or two of the four nucleotides (heterozygous case) is incorporated. The base that is incorporated is complementary to the nucleotide at the SNP position.

A number of methods currently used for SNP detection involve site-specific and/or allele-specific hybridisation (Matsuzaki, H. et al. Genome Res. 14:414-425 (2004); Matsuzaki, H. et al. Nat. Methods 1:109-111 (2004); Sethi, A. A. et al. Clin. Chem. 50(2):443-446 (2004), each of the foregoing is herein incorporated by reference in its entirety). These methods are largely reliant on the discriminatory binding of oligonucleotides to target sequences containing the SNP of interest. The techniques of Affymetrix (Santa Clara, Calif.) and Nanogen Inc. (San Diego, Calif.) are particularly well-known, and utilize the fact that DNA duplexes containing single base mismatches are much less stable than duplexes that are perfectly base-paired. The presence of a matched duplex is detected by fluorescence.

The majority of methods to detect or identify SNPs by site-specific hybridisation require target amplification by methods such as PCR to increase sensitivity and specificity (see, for example U.S. Pat. No. 5,679,524, PCT publication WO 98/59066, PCT publication WO 95/12607, each of the foregoing is herein incorporated by reference in its entirety). US Application 20050059030 (herein incorporated by reference in its entirety) describes a method for detecting a single nucleotide polymorphism in total human DNA without prior amplification or complexity reduction to selectively enrich for the target sequence, and without the aid of any enzymatic reaction. The method utilises a single-step hybridization involving two hybridization events: hybridization of a first portion of the target sequence to a capture probe, and hybridization of a second portion of said target sequence to a detection probe. Both hybridization events happen in the same reaction, and the order in which hybridisation occurs is not critical.

US Application 20050042608 (herein incorporated by reference in its entirety) describes a modification of the method of electrochemical detection of nucleic acid hybridization of Thorp et al. (U.S. Pat. No. 5,871,918, herein incorporated by reference in its entirety). Briefly, capture probes are designed, each of which has a different SNP base and a sequence of probe bases on each side of the SNP base. The probe bases are complementary to the corresponding target sequence adjacent to the SNP site. Each capture probe is immobilized on a different electrode having a non-conductive outer layer on a conductive working surface of a substrate. The extent of hybridization between each capture probe and the nucleic acid target is detected by detecting the oxidation-reduction reaction at each electrode, utilizing a transition metal complex. These differences in the oxidation rates at the different electrodes are used to determine whether the selected nucleic acid target has a single nucleotide polymorphism at the selected SNP site.

The technique of Lynx Therapeutics (Hayward, Calif.) using MEGATYPE™ technology can genotype very large numbers of SNPs simultaneously from small or large pools of genomic material. This technology uses fluorescently labeled probes and compares the collected genomes of two populations, enabling detection and recovery of DNA fragments spanning SNPs that distinguish the two populations, without requiring prior SNP mapping or knowledge.

A number of other methods for detecting and identifying SNPs exist. These include the use of mass spectrometry, for example, to measure probes that hybridize to the SNP (Ross, P. L. et al. Discrimination of single-nucleotide polymorphisms in human DNA using peptide nucleic acid probes detected by MALDI-TOF mass spectrometry. Anal. Chem. 69, 4197-4202 (1997), herein incorporated by reference in its entirety). This technique varies in how rapidly it can be performed, from a few samples per day to a high throughput of 40,000 SNPs per day, using mass code tags. A preferred example is the use of mass spectrometric determination of a nucleic acid sequence which includes the polymorphisms of the invention, for example, which includes the promoter of the COX2 gene or a complementary sequence. Such mass spectrometric methods are known to those skilled in the art, and the genotyping methods of the invention are amenable to adaptation for the mass spectrometric detection of the polymorphisms of the invention, for example, the COX2 promoter polymorphisms of the invention.

SNPs can also be determined by ligation-bit analysis. This analysis requires two primers that hybridize to a target with a one nucleotide gap between the primers. Each of the four nucleotides is added to a separate reaction mixture containing DNA polymerase, ligase, target DNA and the primers. The polymerase adds a nucleotide to the 3′end of the first primer that is complementary to the SNP, and the ligase then ligates the two adjacent primers together. Upon heating of the sample, if ligation has occurred, the now larger primer will remain hybridized and a signal, for example, fluorescence, can be detected. A further discussion of these methods can be found in U.S. Pat. Nos. 5,919,626; 5,945,283; 5,242,794; and 5,952,174 (each of the foregoing is herein incorporated by reference in its entirety).

U.S. Pat. No. 6,821,733 (herein incorporated by reference in its entirety) describes methods to detect differences in the sequence of two nucleic acid molecules that includes the steps of: contacting two nucleic acids under conditions that allow the formation of a four-way complex and branch migration; contacting the four-way complex with a tracer molecule and a detection molecule under conditions in which the detection molecule is capable of binding the tracer molecule or the four-way complex; and determining binding of the tracer molecule to the detection molecule before and after exposure to the four-way complex. Competition of the four-way complex with the tracer molecule for binding to the detection molecule indicates a difference between the two nucleic acids.

Protein- and proteomics-based approaches are also suitable for polymorphism detection and analysis. Polymorphisms which result in or are associated with variation in expressed proteins can be detected directly by analyzing said proteins. This typically requires separation of the various proteins within a sample, by, for example, gel electrophoresis or HPLC, and identification of said proteins or peptides derived therefrom, for example by NMR or protein sequencing such as chemical sequencing or more prevalently mass spectrometry. Proteomic methodologies are well known in the art, and have great potential for automation. For example, integrated systems, such as the ProteomIQ™ system from Proteome Systems, provide high throughput platforms for proteome analysis combining sample preparation, protein separation, image acquisition and analysis, protein processing, mass spectrometry and bioinformatics technologies.

The majority of proteomic methods of protein identification utilise mass spectrometry, including ion trap mass spectrometry, liquid chromatography (LC) and LC/MSn mass spectrometry, gas chromatography (GC) mass spectroscopy, Fourier transform-ion cyclotron resonance-mass spectrometer (FT-MS), MALDI-TOF mass spectrometry, and ESI mass spectrometry, and their derivatives. Mass spectrometric methods are also useful in the determination of post-translational modification of proteins, such as phosphorylation or glycosylation, and thus have utility in determining polymorphisms that result in or are associated with variation in post-translational modifications of proteins.

Associated technologies are also well known, and include, for example, protein processing devices such as the “Chemical Inkjet Printer” including piezoelectric printing technology that allows in situ enzymatic or chemical digestion of protein samples electroblotted from 2-D PAGE gels to membranes by jetting the enzyme or chemical directly onto the selected protein spots (Sloane, A. J. et al. High throughput peptide mass fingerprinting and protein macroarray analysis using chemical printing strategies. Mol Cell Proteomics 1(7):490-9 (2002), herein incorporated by reference in its entirety). After in-situ digestion and incubation of the proteins, the membrane can be placed directly into the mass spectrometer for peptide analysis.

A large number of methods reliant on the conformational variability of nucleic acids have been developed to detect SNPs.

For example, Single Strand Conformational Polymorphism (SSCP, Orita et al., PNAS 86:2766-2770, (1989), herein incorporated by reference in its entirety) is a method reliant on the ability of single-stranded nucleic acids to form secondary structure in solution under certain conditions. The secondary structure depends on the base composition and can be altered by a single nucleotide substitution, causing differences in electrophoretic mobility under nondenaturing conditions. The various polymorphs are typically detected by autoradiography when radioactively labelled, by silver staining of bands, by hybridisation with detectably labelled probe fragments or the use of fluorescent PCR primers which are subsequently detected, for example by an automated DNA sequencer.

Modifications of SSCP are well known in the art, and include the use of differing gel running conditions, such as for example differing temperature, or the addition of additives, and different gel matrices. Other variations on SSCP are well known to the skilled artisan, including, RNA-SSCP (Gasparini, P. et al. Scanning the first part of the neurofibromatosis type 1 gene by RNA-SSCP: identification of three novel mutations and of two new polymorphisms. Hum Genet. 97(4):492-5 (1996), herein incorporated by reference in its entirety), restriction endonuclease fingerprinting-SSCP (Liu, Q. et al. Restriction endonuclease fingerprinting (REF): a sensitive method for screening mutations in long, contiguous segments of DNA. Biotechniques 18(3):470-7 (1995), herein incorporated by reference in its entirety), dideoxy fingerprinting (a hybrid between dideoxy sequencing and SSCP) (Sarkar, G. et al. Dideoxy fingerprinting (ddF): a rapid and efficient screen for the presence of mutations. Genomics 13:441-443 (1992), herein incorporated by reference in its entirety), bi-directional dideoxy fingerprinting (in which the dideoxy termination reaction is performed simultaneously with two opposing primers) (Liu, Q. et al. Bi-directional dideoxy fingerprinting (Bi-ddF): a rapid method for quantitative detection of mutations in genomic regions of 300-600 bp. Hum Mol Genet. 5(1):107-14 (1996), herein incorporated by reference in its entirety), and Fluorescent PCR-SSCP (in which PCR products are internally labelled with multiple fluorescent dyes, can be digested with restriction enzymes, followed by SSCP, and analyzed on an automated DNA sequencer able to detect the fluorescent dyes) (Makino, R. et al. F-SSCP: fluorescence-based polymerase chain reaction-single-strand conformation polymorphism (PCR-SSCP) analysis. PCR Methods Appl. 2(1):10-13 (1992), herein incorporated by reference in its entirety).

Other methods which utilise the varying mobility of different nucleic acid structures include Denaturing Gradient Gel Electrophoresis (DGGE) (Cariello, N. F. et al. Resolution of a missense mutant in human genomic DNA by denaturing gradient gel electrophoresis and direct sequencing using in vitro DNA amplification: HPRT Munich. Am J Hum Genet. 42(5):726-34 (1988), herein incorporated by reference in its entirety), Temperature Gradient Gel Electrophoresis (TGGE) (Riesner, D. et al. Temperature-gradient gel electrophoresis for the detection of polymorphic DNA and for quantitative polymerase chain reaction. Electrophoresis. 13:632-6 (1992), herein incorporated by reference in its entirety), and Heteroduplex Analysis (HET) (Keen, J. et al. Rapid detection of single base mismatches as heteroduplexes on Hydrolink gels. Trends Genet. 7(1):5 (1991), herein incorporated by reference in its entirety). Here, variation in the dissociation of double stranded DNA (for example, due to base-pair mismatches) results in a change in electrophoretic mobility. These mobility shifts are used to detect nucleotide variations.

Denaturing High Pressure Liquid Chromatography (HPLC) is yet a further method utilised to detect SNPs, using HPLC methods well-known in the art as an alternative to the separation methods described above (such as gel electophoresis) to detect, for example, homoduplexes and heteroduplexes which elute from the HPLC column at different rates, thereby enabling detection of mismatch nucleotides and thus SNPs (Giordano, M. et al. Identification by denaturing high-performance liquid chromatography of numerous polymorphisms in a candidate region for multiple sclerosis susceptibility. Genomics 56(3):247-53 (1999), herein incorporated by reference in its entirety).

Yet further methods to detect SNPs rely on the differing susceptibility of single stranded and double stranded nucleic acids to cleavage by various agents, including chemical cleavage agents and nucleolytic enzymes. For example, cleavage of mismatches within RNA:DNA heteroduplexes by RNase A, of heteroduplexes by, for example bacteriophage T4 endonuclease YII or T7 endonuclease I, of the 5′ end of the hairpin loops at the junction between single stranded and double stranded DNA by cleavase I, and the modification of mispaired nucleotides within heteroduplexes by chemical agents commonly used in Maxam-Gilbert sequencing chemistry, are all well known in the art.

Further examples include the Protein Translation Test (PTT), used to resolve stop codons generated by variations which lead to a premature termination of translation and to protein products of reduced size, and the use of mismatch binding proteins (Moore, W. et al. Mutation detection in the breast cancer gene BRCA1 using the protein truncation test. Mol Biotechnol. 14(2):89-97 (2000), herein incorporated by reference in its entirety). Variations are detected by binding of, for example, the MutS protein, a coimponent of Escherichia coli DNA mismatch repair system, or the human hMSH2 and GTBP proteins, to double stranded DNA heteroduplexes containing mismatched bases. DNA duplexes are then incubated with the mismatch binding protein, and variations are detected by mobility shift assay. For example, a simple assay is based on the fact that the binding of the mismatch binding protein to the heteroduplex protects the heteroduplex from exonuclease degradation.

Those skilled in the art will know that a particular SNP, particularly when it occurs in a regulatory region of a gene such as a promoter, can be associated with altered expression of a gene. Altered expression of a gene can also result when the SNP is located in the coding region of a protein-encoding gene, for example where the SNP is associated with codons of varying usage and thus with tRNAs of differing abundance. Such altered expression can be determined by methods well known in the art, and can thereby be employed to detect such SNPs. Similarly, where a SNP occurs in the coding region of a gene and results in a non-synonomous amino acid substitution, such substitution can result in a change in the function of the gene product. Similarly, in cases where the gene product is an RNA, such SNPs can result in a change of function in the RNA gene product. Any such change in function, for example as assessed in an activity or functionality assay, can be employed to detect such SNPs.

The above methods of detecting and identifying SNPs are amenable to use in the methods of the invention.

Of course, in order to detect and identify SNPs in accordance with the invention, a sample containing material to be tested is obtained from the subject. The sample can be any sample potentially containing the target SNPs (or target polypeptides, as the case may be) and obtained from any bodily fluid (blood, urine, saliva, etc) biopsies or other tissue preparations.

DNA or RNA can be isolated from the sample according to any of a number of methods well known in the art. For example, methods of purification of nucleic acids are described in Tijssen; Laboratory Techniques in Biochemistry and Molecular Biology: Hybridization with nucleic acid probes Part 1: Theory and Nucleic acid preparation, Elsevier, New York, N.Y. 1993, as well as in Maniatis, T., Fritsch, E. F. and Sambrook, J., Molecular Cloning Manual 1989 (each of the foregoing is herein incorporated by reference in its entirety).

To assist with detecting the presence or absence of polymorphisms/SNPs, nucleic acid probes and/or primers can be provided. Such probes have nucleic acid sequences specific for chromosomal changes evidencing the presence or absence of the polymorphism and are preferably labeled with a substance that emits a detectable signal when combined with the target polymorphism.

The nucleic acid probes can be genomic DNA or cDNA or mRNA, or any RNA-like or DNA-like material, such as peptide nucleic acids, branched DNAs, and the like. The probes can be sense or antisense polynucleotide probes. Where target polynucleotides are double-stranded, the probes can be either sense or antisense strands. Where the target polynucleotides are single-stranded, the probes are complementary single strands.

The probes can be prepared by a variety of synthetic or enzymatic schemes, which are well known in the art. The probes can be synthesized, in whole or in part, using chemical methods well known in the art (Caruthers et al., Nucleic Acids Res., Symp. Ser., 215-233 (1980), herein incorporated by reference in its entirety). Alternatively, the probes can be generated, in whole or in part, enzymatically.

Nucleotide analogs can be incorporated into probes by methods well known in the art. The only requirement is that the incorporated nucleotide analog must serve to base pair with target polynucleotide sequences. For example, certain guanine nucleotides can be substituted with hypoxanthine, which base pairs with cytosine residues. However, these base pairs are less stable than those between guanine and cytosine. Alternatively, adenine nucleotides can be substituted with 2,6-diaminopurine, which can form stronger base pairs than those between adenine and thymidine.

Additionally, the probes can include nucleotides that have been derivatized chemically or enzymatically. Typical chemical modifications include derivatization with acyl, alkyl, aryl or amino groups.

The probes can be immobilized on a substrate. Preferred substrates are any suitable rigid or semi-rigid support including membranes, filters, chips, slides, wafers, fibers, magnetic or nonmagnetic beads, gels, tubing, plates, polymers, microparticles and capillaries. The substrate can have a variety of surface forms, such as wells, trenches, pins, channels and pores, to which the polynucleotide probes are bound. Preferably, the substrates are optically transparent.

Furthermore, the probes do not have to be directly bound to the substrate, but rather can be bound to the substrate through a linker group. The linker groups are typically about 6 to 50 atoms long to provide exposure to the attached probe. Preferred linker groups include ethylene glycol oligomers, diamines, diacids and the like. Reactive groups on the substrate surface react with one of the terminal portions of the linker to bind the linker to the substrate. The other terminal portion of the linker is then functionalized for binding the probe.

The probes can be attached to a substrate by dispensing reagents for probe synthesis on the substrate surface or by dispensing preformed DNA fragments or clones on the substrate surface. Typical dispensers include a micropipette delivering solution to the substrate with a robotic system to control the position of the micropipette with respect to the substrate. There can be a multiplicity of dispensers so that reagents can be delivered to the reaction regions simultaneously.

Nucleic acid microarrays are preferred. Such microarrays (including nucleic acid chips) are well known in the art (see, for example U.S. Pat. Nos. 5,578,832; 5,861,242; 6,183,698; 6,287,850; 6,291,183; 6,297,018; 6,306,643; and 6,308,170, each of the foregoing is herein incorporated by reference in its entirety).

Alternatively, antibody microarrays can be produced. The production of such microarrays is essentially as described in Schweitzer & Kingsmore, “Measuring proteins on microarrays”, Curr Opin Biotechnol 2002; 13(1): 14-9; Avseekno et al., “Immobilization of proteins in immunochemical microarrays fabricated by electrospray deposition”, Anal Chem 2001 15; 73(24): 6047-52; Huang, “Detection of multiple proteins in an antibody-based protein microarray system, Immunol Methods 2001 1; 255 (1-2): 1-13 (each of the foregoing is herein incorporated by reference in its entirety).

The present invention also contemplates the preparation of kits for use in accordance with the present invention. Suitable kits include various reagents for use in accordance with the present invention in suitable containers and packaging materials, including tubes, vials, and shrink-wrapped and blow-molded packages.

Materials suitable for inclusion in an exemplary kit in accordance with the present invention include one or more of the following: gene specific PCR primer pairs (oligonucleotides) that anneal to DNA or cDNA sequence domains that flank the genetic polymorphisms of interest, reagents capable of amplifying a specific sequence domain in either genomic DNA or cDNA without the requirement of performing PCR; reagents required to discriminate between the various possible alleles in the sequence domains amplified by PCR or non-PCR amplification (e.g., restriction endonucleases, oligonucleotide that anneal preferentially to one allele of the polymorphism, including those modified to contain enzymes or fluorescent chemical groups that amplify the signal from the oligonucleotide and make discrimination of alleles more robust); reagents required to physically separate products derived from the various alleles (e.g. agarose or polyacrylamide and a buffer to be used in electrophoresis, HPLC columns, SSCP gels, formamide gels or a matrix support for MALDI-TOF).

It will be appreciated that the methods of the invention can be performed in conjunction with an analysis of other risk factors known to be associated with OCOPD. Such risk factors include epidemiological risk factors associated with an increased risk of developing OCOPD. Such risk factors include, but are not limited to smoking and/or exposure to tobacco smoke, age, sex and familial history. These risk factors can be used to augment an analysis of one or more polymorphisms as herein described when assessing a subject's risk of developing chronic obstructive pulmonary disease (COPD) and/or emphysema.

The predictive methods of the invention allow a number of therapeutic interventions and/or treatment regimens to be assessed for suitability and implemented for a given subject. The simplest of these can be the provision to the subject of motivation to implement a lifestyle change, for example, where the subject is a current smoker, the methods of the invention can provide motivation to quit smoking.

The manner of therapeutic intervention or treatment will be predicated by the nature of the polymorphism(s) and the biological effect of said polymorphism(s). For example, where a susceptibility polymorphism is associated with a change in the expression of a gene, intervention or treatment is preferably directed to the restoration of normal expression of said gene, by, for example, administration of an agent capable of modulating the expression of said gene. Where a polymorphism is associated with decreased expression of a gene, therapy can involve administration of an agent capable of increasing the expression of said gene, and conversely, where a polymorphism is associated with increased expression of a gene, therapy can involve administration of an agent capable of decreasing the expression of said gene. Methods useful for the modulation of gene expression are well known in the art. For example, in situations where a polymorphism is associated with upregulated expression of a gene, therapy utilising, for example, RNAi or antisense methodologies can be implemented to decrease the abundance of mRNA and so decrease the expression of said gene. Alternatively, therapy can involve methods directed to, for example, modulating the activity of the product of said gene, thereby compensating for the abnormal expression of said gene.

Where a susceptibility polymorphism is associated with decreased gene product function or decreased levels of expression of a gene product, therapeutic intervention or treatment can involve augmenting or replacing of said function, or supplementing the amount of gene product within the subject for example, by administration of said gene product or a functional analogue thereof. For example, where a polymorphism is associated with decreased enzyme function, therapy can involve administration of active enzyme or an enzyme analogue to the subject. Similarly, where a polymorphism is associated with increased gene product function, therapeutic intervention or treatment can involve reduction of said function, for example, by administration of an inhibitor of said gene product or an agent capable of decreasing the level of said gene product in the subject. For example, where a SNP allele or genotype is associated with increased enzyme function, therapy can involve administration of an enzyme inhibitor to the subject.

Likewise, when a protective polymorphism is associated with upregulation of a particular gene or expression of an enzyme or other protein, therapies can be directed to mimic such upregulation or expression in an individual lacking the resistive genotype, and/or delivery of such enzyme or other protein to such individual Further, when a protective polymorphism is associated with downregulation of a particular gene, or with diminished or eliminated expression of an enzyme or other protein, desirable therapies can be directed to mimicking such conditions in an individual that lacks the protective genotype.

The relationship between the various polymorphisms identified above and the susceptibility (or otherwise) of a subject to OCOPD also has application in the design and/or screening of candidate therapeutics. This is particularly the case where the association between a susceptibility or protective polymorphism is manifested by either an upregulation or downregulation of expression of a gene. In such instances, the effect of a candidate therapeutic on such upregulation or downregulation is readily detectable.

For example, in one embodiment existing human lung organ and cell cultures are screened for SNP genotypes as set forth above. (For information on human lung organ and cell cultures, see, e.g.: Bohinski et al. (1996) Molecular and Cellular Biology 14:5671-5681; Collettsolberg et al. (1996) Pediatric Research 39:504; Hermanns et al. (2004) Laboratory Investigation 84:736-752; Hume et al. (1996) In Vitro Cellular & Developmental Biology-Animal 32:24-29; Leonardi et al. (1995) 38:352-355; Notingher et al. (2003) Biopolymers (Biospectroscopy) 72:230-240; Ohga et al. (1996) Biochemical and Biophysical Research Communications 228:391-396; each of the foregoing is herein incorporated by reference in its entirety.) Cultures representing susceptibility and protective genotype groups are selected, together with cultures which are putatively “normal” in terms of the expression of a gene which is either upregulated or downregulated where a protective polymorphism is present.

Samples of such cultures are exposed to a library of candidate therapeutic compounds and screened for any or all of: (a) downregulation of susceptibility genes that are normally upregulated in susceptibility genotypes; (b) upregulation of susceptibility genes that are normally downregulated in susceptibility genotypes; (c) downregulation of protective genes that are normally downregulated or not expressed (or null forms are expressed) in protective genotypes; and (d) upregulation of protective genes that are normally upregulated in protective genotypes. Compounds are selected for their ability to alter the regulation and/or action of susceptibility genes and/or protective genes in a culture having a susceptibility genotype.

Similarly, where the polymorphism is one which when present results in a physiologically active concentration of an expressed gene product outside of the normal range for a subject (adjusted for age and sex), and where there is an available prophylactic or therapeutic approach to restoring levels of that expressed gene product to within the normal range, individual subjects can be screened to determine the likelihood of their benefiting from that restorative approach. Such screening involves detecting the presence or absence of the polymorphism in the subject by any of the methods described herein, with those subjects in which the polymorphism is present being identified as individuals likely to benefit from treatment.

The invention will now be described in more detail, with reference to non-limiting examples.

### EXAMPLE 1

### Case Association Study

Methods

Subject Recruitment

Subjects of European decent who had been exposed to chronic smoking (minimum 15 pack years) and aero-pollutants in the work place (noxious dusts or fumes) were identified from respiratory clinics. After spirometric testing we recruited those with chronic obstructive pulmonary disease (COPD) with forced expiratory volume in one second (FEV1) as a percentage of predicted <70% and a FEV1/FVC ratio (Forced expiratory volume in one second/Forced vital capacity) of <79% (measured using American Thoracic Society criteria). One hundred and thirty-nine subjects were recruited, of these 70% were male, the mean FEV1/FVC (±Standard Deviation) was 54% (SD 15), mean FEV1 as a percentage of predicted was 46 (SD 19). Mean age, cigarettes per day and pack year history was 62 yrs (SD 9), 25 cigarettes/day (SD 16) and 53 pack years (SD 31) respectively. We also studied one hundred and twelve European subjects who had smoked a minimum of fifteen pack years and similarly been exposed in the work place to potentially noxious dusts or fumes. This control group was recruited through community studies of lung function and included 81% male, the mean FEV1/FVC (SD) was 81% (SD 8), mean FEV1 as a percentage of predicted was 96 (SD 10). Mean age, cigarettes per day and pack year history was 58 yrs (SD 11), 26 cigarettes/day (SD 14) and 45 pack years (SD 28) respectively. Using a PCR based method (Sandford et al., 1999 [6, herein incorporated by reference in its entirety]), we genotyped all subjects were genotype for the α1-antitrypsin mutations (M, S and Z alleles) and had excluded those with the ZZ allele were excluded. The COPD and resistant smoker cohorts were matched for subjects with the MZ genotype (6% in each cohort). They were also matched for age started smoking (mean 16 yr) and aged stopped smoking (mid fifties). 190 European blood donors (smoking and occupational exposure status unknown), were recruited consecutively through local blood donor services. Sixty-three percent were men and their mean age was 50 years. On regression analysis, the age difference and pack years difference observed between OCOPD sufferers and resistant smokers was found not to determine FEV or OCOPD.

TABLE 1C
Summary Of Characteristics For The OCOPD Subjects And
Resistant Smokers With Occupational Exposure.

| | Aero-pollutant | | |
|---|---|---|---|
| Parameter | exposed | Exposed resistant | |
| Mean (SD) | COPD (N = 139) | smokers (N = 112) | Differences |
| % male | 70% | 81% | P < 0.05 |
| Age (yrs) | 62 (9) | 58 (11) | ns |
| Pack years | 53 (31) | 45 (28) | P < 0.05 |
| Cigarettes/day | 25 (16) | 26 (14) | ns |
| FEV1 (L) | 1.3 (0.7) | 3.0 (0.7) | P < 0.05 |
| FEV1 % predict | 46 (19) | 96% (10) | P < 0.05 |
| FEV1/FVC | 54 (15) | 81 (8) | P < 0.05 |

Means and 1SD
Genotyping Methods
Cyclo-oxygenase 2 (COX2)-765 G/C Promoter Polymorphism and α1-antitrypsin genotyping.

Genomic DNA was extracted from whole blood samples [Maniatis, T., Fritsch, E. F. and Sambrook, J., Molecular Cloning Manual. 1989 [7, herein incorporated by reference in its entirety]]. The Cyclo-oxygenase 2-765 polymorphism was determined by minor modifications of a previously published method [Papafili A, et al, 2002, [8, herein incorporated by reference in its entirety]]. The PCR reaction was carried out in a total volume of 25ul and contained 20 ng genomic DNA, 500 pmol forward and reverse primers, 0.2 mM dNTPs, 10 mM Tris-HCL (pH 8.4), 150 mM KCl, 1.0 mM MgCl₂ and 1 unit of Taq polymerase (Life Technologies). Cycling times were incubations for 3 mins at 95° C. followed by 33 cycles of 50s at 94° C., 60s at 66° C. and 60s at 72° C. A final elongation of 10 mins at 72° C. then followed. 4 ul of PCR products were visualised by ultraviolet trans-illumination of a 6% agarose gel stained with ethidium bromide. An aliquot of 3 ul of amplification product was digested for 1 hr with 4 units of Aci I (Roche Diagnostics, New Zealand) at 37° C. Digested products were separated on a 2.5% agarose gel run for 2.0 hrs at 80 mV with TBE buffer. Using ultraviolet transillumination after ethidium bromide staining. The products were visualised against a 123 bp ladder. Using a PCR based method referenced above [Sandford et al., 1999[6]], all smoking subjects were genotyped for the α1-antitrypsin M, S and Z alleles.

Genotyping of the Superoxide Dismutase 3 Arg 312 Gln polymorphism

Genomic DNA was extracted using standard phenol and chloroform methods. Cohorts of patients and controls were configured in to 96-well PCR format containing strategic negative controls. The assay primers, PCR conditions and RFLP assays details have been previously described [9, herein incorporated by reference in its entirety]. Genotyping was done using minor modifications of the above protocol optimised for laboratory conditions. The PCR reactions were amplified in MJ Research thermocyclers in a total volume of 25 μl and contained 80 ng genomic DNA, 10 pmol forward and reverse primers, 0.1 mM dNTPs, 10 mM Tris-HCL (pH 8.4), 150 mM KCl, 1.0 mM MgCl₂ and 0.5 unit of Taq polymerase (Qiagen). Aliquots of amplification product were digested for 4 hrs with 5Units of the relevant restriction enzymes (Roche Diagnostics, New Zealand) at designated temperature conditions. Digested products were separated on an 8% polyacrylamide gels (49:1, Sigma). The products were visualized by ultraviolet transillumination following ethidium bromide staining and migration compared against a 1 Kb plus ladder standard (Invitrogen). Genotypes were recorded in data spreadsheets and statistical analysis performed.

Genotyping of the Microsomal Epoxide Hydrolase Exon 3 TC Polymorphism

Genomic DNA was extracted using standard phenol and chloroform methods. Cohorts of patients and controls were configured in to 96-well PCR format containing strategic negative controls. The assay primers, PCR conditions and RFLP assays details have been previously described [Smith et al. [10, herein incorporated by reference in its entirety]]. Genotyping was done using minor modifications of the above protocol optimized for laboratory conditions The PCR reactions were amplified in MJ Research thermocyclers in a total volume of 2511 and contained 80 ng genomic DNA, 100 ng forward and reverse primers, 0.2 mM dNTPs, 10 mM Tris-HCL (pH 8.4), 150 mM KCl, 1.5 mM MgCl₂ and 1.0 unit of Taq polymerase (Qiagen). Cycling conditions consisted of 94° C. 60 s, 56° C. 20 s, 72° C. 20 s for 38 cycles with an extended last extension of 3 min. Aliquots of amplification product were digested for 4 hrs with 5Units of the relevant restriction enzymes Eco RV (Roche Diagnostics, New Zealand) at designated temperature conditions. Digested products were separated on an 8% polyacrylamide gels (49:1, Sigma). The products were visualized by ultraviolet transillumination following ethidium bromide staining and migration compared against a 1 Kb plus ladder standard (Invitrogen). Genotypes were recorded in data spreadsheets and statistical analysis performed.

Genotyping of the 3′1237 G/A (T/t) Polymorphism of the α1-Antitrypsin Gene

Genomic DNA was extracted using standard phenol and chloroform methods. Cohorts of patients and controls were configured in to 96-well PCR format containing strategic negative controls. The assay primers, PCR conditions and RFLP assays details have been previously described [Sandford A J et al.[6]]. Genotyping was done using minor modifications of the above protocol optimized for laboratory conditions The PCR reactions were amplified in MJ Research thermocyclers in a total volume of 2511 and contained 80 ng genomic DNA, 100 ng forward and reverse primers, 0.2 mM dNTPs, 10 mM Tris-HCL (pH 8.4), 150 mM KCl, 1.5 mM MgCl₂ and 1.0 unit of Taq polymerase (Qiagen). Forward and reverse prime sequences were 5′-CTACCAGGAATGGCCTTGTCC 3′ [SEQ. ID. NO.1] and 5′-CTCTCAGGTCTGGTGTCATCC 3′ [SEQ. ID. NO.2]. Cycling conditions consisted of 94° C. 60 s, 56° C. 20 s, 72° C. 20 s for 38 cycles with an extended last extension of 3 min. Aliquots of amplification product were digested for 4 hrs with 2 Units of the restriction enzymes Taq 1 (Roche Diagnostics, New Zealand) at designated temperature conditions. Digested products were separated on 3% agarose. The products were visualized by ultraviolet transillumination following ethidium bromide staining and migration compared against a 1 Kb plus ladder standard (Invitrogen). Genotypes were recorded in data spreadsheets and statistical analysis performed.

Genotyping of the Asp 299 Gly Polymorphism of the Toll-like Receptor 4 Gene

Genomic DNA was extracted using standard phenol and chloroform methods. Cohorts of patients and controls were configured in to 96-well PCR format containing strategic negative controls. The assay primers, PCR conditions and RFLP assays details have been previously described [Lorenz E, et al., [11, herein incorporated by reference in its entirety]]. Genotyping was done using minor modifications of the above protocol optimized for laboratory conditions The PCR reactions were amplified in MJ Research thermocyclers in a total volume of 25 μl and contained 80 ng genomic DNA, 100 ng forward and reverse primers, 0.2 mM dNTPs, 10 mM Tris-HCL (pH 8.4), 150 mM KCl, 1.5 mM MgCl₂ and 1.0 unit of Taq polymerase (Qiagen). Forward and reverse prime sequences were 5′-GATTAGCATACTTAGACTACTACCTCCATG-3′ [SEQ. ID. NO.3] and 5′-GATCAACTTCTGAAAAAGCATTCCCAC-3′ [SEQ. ID. NO.4]. Cycling conditions consisted of 94C 30 s, 55C 30 s, 72C 30 s for 30 cycles with an extended last extension of 3 min. Aliquots of amplification product were digested for 4 hrs with 2 Units of the restriction enzyme Nco I (Roche Diagnostics, New Zealand) at designated temperature conditions. Digested products were separated on 3% agarose gel. The products were visualized by ultraviolet transillumination following ethidium bromide staining and migration compared against a 1 Kb plus ladder standard (Invitrogen). Genotypes were recorded in data spreadsheets and statistical analysis performed.

Genotyping of the −1607 1G2G Polymorphism of the Matrix Metalloproteinase 1 Gene

Genomic DNA was extracted using standard phenol and chloroform methods. Cohorts of patients and controls were configured in to 96-well PCR format containing strategic negative controls. The assay primers, PCR conditions and RFLP assays details have been previously described [Dunleavey L, et al. Rapid genotype analysis of the matrix metalloproteinase-1 gene 1G/2G polymorphism that is associated with risk of cancer. Matrix Biol. 19(2): 175-7 (2000), herein incorporated by reference in its entirety]. Genotyping was done using minor modifications of the above protocol optimized for laboratory conditions The PCR reactions were amplified in MJ Research thermocyclers in a total volume of 25 μl and contained 80 ng genomic DNA, 100 ng forward and reverse primers, 200 mM dNTPs, 20 mM Tris-HCL (pH 8.4), 50 mM KCl, 1.5 mM MgCl₂ and 1.0 unit of Taq polymerase (Qiagen). Forward and reverse prime sequences were 3′TCGTGAGAATGTCTTCCCATT-3′ [SEQ. ID. NO.5] and 5′-TCTTGGATTGATTTGAGATAAGTGAAATC-3′ [SEQ. ID. NO.6]. Cycling conditions consisted of 94° C. 60 s, 55° C. 30 s, 72° C. 30 s for 35 cycles with an extended last extension of 3 min. Aliquots of amplification product were digested for 4 hrs with 6 Units of the restriction enzymes XmnI (Roche Diagnostics, New Zealand) at designated temperature conditions. Digested products were separated on 6% polyacrylamide gel. The products were visualized by ultraviolet transillumination following ethidium bromide staining and migration compared against a 1 Kb plus ladder standard (Invitrogen). Genotypes were recorded in data spreadsheets and statistical analysis performed.

Other Polymorphism Genotyping.

Genomic DNA was extracted from whole blood samples (Maniatis, T., Fritsch, E. F. and Sambrook, J., Molecular Cloning Manual. 1989[7]). Purified genomic DNA was aliquoted (10 ng/ul concentration) into 96 well plates and genotyped on a Sequenom™ system (Sequenom™ Autoflex Mass Spectrometer and Samsung 24 pin nanodispenser) using the following sequences, amplification conditions and methods.

The following conditions were used for the PCR multiplex reaction: final concentrations were for 10× Buffer 15 mM MgCl2 1.25x, 25 mM MgCl2 1.625 mM, dNTP mix 25 mM 500 uM, primers 4 uM 100 nM, Taq polymerase (Quiagen hot start) 0.15u/reaction, Genomic DNA 10 ng/ul. Cycling times were 95° C. for 15 min, (5° C. for 15 s, 56° C. 30 s, 72° C. 30 s for 45 cycles with a prolonged extension time of 3 min to finish. Shrimp alkaline phosphotase (SAP) treatment was used, (2 ul to 5 ul PCR reaction) incubated at 35° C. for 30 min and extension reaction (add 2 ul to 7 ul after SAP treatment) with the following volumes per reaction of water 0.76 ul, hME 10× termination buffer 0.2 ul, hME primer (10 uM) 1 ul, MassEXTEND enzyme 0.04 ul.

TABLE 1D

Sequenom conditions for the polymorphisms genotyping-1

| | | | | |
|---|---|---|---|---|
| SNP_ID | TERM | WELL | 2nd-PCRP | 1st-PCRP |

| | | | | |
|---|---|---|---|---|
| Vitamin DBP- | ACT | W1 | ACGTTGGATGGCTTGTTAACCA | ACGTTGGATGTTTTTCAGACTG |
| 420 | | | GCTTTGCC | GCAGAGCG |
| | | | [SEQ. ID. NO. 7] | [SEQ. ID. NO. 8] |
| Vitamin DBP- | ACT | W1 | ACGTTGGATGTTTTTCAGACTG | ACGTTGGATGGCTTGTTAACCA |
| 416 | | | GCAGAGCG | GCTTTGCC |
| | | | [SEQ. ID. NO. 9] | [SEQ. ID. NO. 10] |
| ADRB2- | ACT | W2 | ACGTTGGATGTTGCTGGCACCC | ACGTTGGATGATGAGAGACATG |
| Gln27Glu | | | AATGGAAG | ACGATGCC |
| | | | [SEQ. ID. NO. 11] | [SEQ. ID. NO. 12] |
| GSTP1-105 | ACT | W2 | ACGTTGGATGTGGTGGACATGG | ACGTTGGATGTGGTGCAGATGC |
| | | | TGAATGAC | TCACATAG |
| | | | [SEQ. ID. NO. 13] | [SEQ. ID. NO. 14] |
| PAI1 G- | ACT | W2 | ACGTTGGATGCACAGAGAGAGT | ACGTTGGATGCTCTTGGTCTTT |
| 675G | | | CTGGACAC | CCCTCATC |
| | | | [SEQ. ID. NO. 15] | [SEQ. ID. NO. 16] |
| IL11 G518A | ACT | W3 | ACGTTGGATGCCTCTGATCCTC | ACGTTGGATGAAGAGGGAGTGG |
| | | | TTTGCTTC | AAGGGAAG |
| | | | [SEQ. ID. NO. 17] | [SEQ. ID. NO. 18] |
| NOS3-298 | ACT | W3 | ACGTTGGATGACAGCTCTGCAT | ACGTTGGATGAGTCAATCCCTT |
| | | | TCAGCACG | TGGTGCTC |
| | | | [SEQ. ID. NO. 19] | [SEQ. ID. NO. 20] |
| IL8 A-251T | CGT | W5 | ACGTTGGATGACTGAAGCTCCA | ACGTTGGATGGCCACTCTAGTA |
| | | | CAATTTGG | CTATATCTG |
| | | | [SEQ. ID. NO. 21] | [SEQ. ID. NO. 22] |
| IL18 C-133G | ACT | W6 | ACGTTGGATGGGGTATTCATAA | ACGTTGGATGCCTTCAAGTTCA |
| | | | GCTGAAAC | GTGGTCAG |
| | | | [SEQ. ID. NO. 23] | [SEQ. ID. NO. 24] |
| IL18 A105C | ACT | W8 | ACGTTGGATGGGTCAATGAAGA | ACGTTGGATGAATGTTTATTGT |
| | | | GAACTTGG | AGAAAACC |
| | | | [SEQ. ID. NO. 25] | [SEQ. ID. NO. 26] |

SEQUENOM CONDITIONS FOR THE POLYMORPHISMS GENOTYPING-2

| | | | | | | | |
|---|---|---|---|---|---|---|---|
| SNP_ID | AMP_LEN | UP_CONF | MP_CONE | Tm (NN) | PcGC | PWARN | UEP_DIR |

| | | | | | | | |
|---|---|---|---|---|---|---|---|
| Vitamin DBP-420 | 99 | 99.7 | 99.7 | 46.2 | 53.3 | ML | R |
| Vitamin DBP-416 | 99 | 99.7 | 99.7 | 45.5 | 33.3 | M | F |
| ADRB2-Gln27Glu | 118 | 96.6 | 80 | 52.2 | 66.7 | L | F |
| GSTP1-105 | 107 | 99.4 | 80 | 49.9 | 52.9 | | F |
| PAI1 G-675G | 109 | 97.9 | 80 | 59.3 | 66.7 | g | F |
| IL11 G518A | 169 | 97.5 | 65 | 52.9 | 52.6 | s | F |
| NOS3-298 | 186 | 98.1 | 65 | 61.2 | 63.2 | | F |
| IL8A-251T | 119 | 92.6 | 81.2 | 45.9 | 28.6 | | R |
| IL18 C-133G | 112 | 93.5 | 74.3 | 41.8 | 46.7 | L | F |
| IL18 A105C | 121 | 67.2 | 74.3 | 48.9 | 40 | | R |

Sequenom conditions for the polymorphisms genotyping-3

| | | | | |
|---|---|---|---|---|
| SNP_ID | UEP_MASS | UEP_SEQ | EXT1_CALL | EXT1_MASS |

| | | | | |
|---|---|---|---|---|
| Vitamin DBP-420 | 4518.9 | AGCTTTGCCAGTTCC | A | 4807.1 |
| | | [SEQ. ID. NO. 27] | | |
| Vitamin DBP-416 | 5524.6 | AAAAGCAAAATTGCCTGA | T | 5812.8 |
| | | [SEQ. ID. NO. 28] | | |
| ADRB2-Gln27Glu | 4547 | CACGACGTCACGCAG | C | 4820.2 |
| | | [SEQ. ID. NO. 29] | | |
| GSTP1-105 | 5099.3 | ACCTCCGCTGCAAATAC | A | 5396.5 |
| | | [SEQ. ID. NO. 30] | | |
| PAI1 G-675G | 5620.6 | GAGTCTGGACACGTGGGG | DEL | 5917.9 |
| | | [SEQ. ID. NO. 31] | | |
| IL11 G518A | 5705.7 | TCCATCTCTGTGGATCTCC | A | 6002.9 |
| | | [SEQ. ID. NO. 32] | | |
| NOS3-298 | 5813.8 | TGCTGCAGGCCCCAGATGA | T | 6102 |
| | | [SEQ. ID. NO. 33] | | |
| IL8 A-251T | 6428.2 | CACAATTTGGTGAATTATCAA | A | 6716.4 |
| | | [SEQ. ID. NO. 34] | | |
| IL18 C-133G | 4592 | AGCTGAAACTTCTGG | C | 4865.2 |
| | | [SEQ. ID. NO. 35] | | |
| IL18 A105C | 6085 | TCAAGCTTGCCAAAGTAATC | A | 6373.2 |
| | | [SEQ. ID. NO. 36] | | |

Sequenom conditions for the polymorphisms genotyping-4

1st

| | | | | | |
|---|---|---|---|---|---|
| SNP_ID | EXT1_SEQ | EXT2_CALL | EXT2_MASS | EXT2_SEQ | PAUSE |

| | | | | | |
|---|---|---|---|---|---|
| Vitamin | AGCTTTGCCAGTTCCT | C | 5136.4 | AGCTTTGCCAGTTCCG | 4848.2 |
| DBP-420 | [SEQ. ID. NO. 37] | | | T | |
| | | | | [SEQ. ID. NO. 38] | |
| Vitamin | AAAAGCAAAATTGCCTGA | G | 6456.2 | AAAAGCAAAATTGCCT | 5853.9 |
| DBP-416 | A | | | GAGGC | |
| | [SEQ. ID. NO. 39] | | | [SEQ. ID. NO. 40] | |
| ADRB2- | CACGACGTCACGCAGC | G | 5173.4 | CACGACGTCACGCAGG | 4876.2 |
| Gln27Glu | [SEQ. ID. NO. 41] | | | A | |
| | | | | [SEQ. ID. NO. 42] | |
| GSTP1- | ACCTCCGCTGCAAATACA | G | 5716.7 | ACCTCCGCTGCAAATA | 5428.5 |
| 105 | [SEQ. ID. NO. 43] | | | CGT | |
| | | | | [SEQ. ID. NO. 44] | |
| PAI1 G- | GAGTCTGGACACGTGGGG | G | 6247.1 | GAGTCTGGACACGTGG | 5949.9 |
| 675G | A | | | GGGA | |
| | [SEQ. ID. NO. 45] | | | [SEQ. ID. NO. 46] | |
| IL11 | TCCATCTCTGTGGATCTC | G | 6323.1 | TCCATCTCTGTGGATC | 6034.9 |
| G518A | CA | | | TCCGT | |
| | [SEQ. ID. NO. 47] | | | [SEQ. ID. NO. 48] | |
| NOS3-298 | TGCTGCAGGCCCCAGATG | G | 6416.2 | TGCTGCAGGCCCCAGA | 6143 |
| | AT | | | TGAGC | |
| | [SEQ. ID. NO. 49] | | | [SEQ. ID. NO. 50] | |
| IL8 | CACAATTTGGTGAATTAT | T | 7029.6 | CACAATTTGGTGAATT | 6741.4 |
| A-251T | CAAT | | | ATCAAAT | |
| | [SEQ. ID. NO. 51] | | | [SEQ. ID. NO. 52] | |
| IL18 | AGCTGAAACTTCTGGC | G | 5218.4 | AGCTGAAACTTCTGGG | 4921.2 |
| C-133G | [SEQ. ID. NO. 53] | | | A | |
| | | | | [SEQ. ID. NO. 54] | |
| IL18 | TCAAGCTTGCCAAAGTAA | C | 7040.6 | TCAAGCTTGCCAAAGT | 6414.2 |
| A105C | TCT | | | AATCGGA | |
| | [SEQ. ID. NO. 55] | | | [SEQ. ID. NO. 56] | |

Results

### EXAMPLE 2

### Cyclo-Oxygenase 2 Polymorphism Allele And Genotype Frequency in the Exposed COPD Patients, Exposed Resistant Smokers and Controls

The genotype frequency for the above allele was determined in exposed COPD patients, exposed resistant smokers, and controls. The frequencies are shown in the following table.

TABLE 1E
Cyclo-Oxygenase 2 Polymorphism Allele
And Genotype Frequencyin The Exposed COPD Patients,
Exposed Resistant Smokers And Controls.

| | Allele* | | Genotype | | |
|---|---|---|---|---|---|
| Frequency | C | G | CC | CG | GG |
| Controls n = 95 (%) | 27 (14%) | 161 (86%) | 3 | 21 | 70 |
| | | | (3%) | (22%) | (75%) |
| Exposed COPD n = 82 | 22 (13%) | 142 (87%) | 2 | 18 | 62¹ |
| (%) | | | (2%) | (22%) | (76%) |
| Exposed Resistant | 42 (24%) | 132 (76%) | 6³ | 30 | 51² |
| n = 87 (%) | | | (7%) | (34%) | (59%) |

*number of chromosomes (2n)

A mathematical analysis of the data in the table indicated that:
1. Genotype. CC/CG vs GG for resistant vs COPD, Odds ratio (OR)=2.2, 95% confidence limits=1.1-4.8, χ² (Yates corrected)=4.76, P=0.03 CC/CG=protective for OCOPD;
2. Allele. C vs G for resistant vs COPD, Odds ratio (OR)=2.1, 95% confidence limits 1.1-3.8, χ² (Yates corrected)=5.65, p=0.02, C=protective for OCOPD;
3. Genotype. GG vs CG/CC for COPD vs resistant, Odds ratio (OR)=0.5, 95% confidence limits=0.2-0.9, χ² (Yates corrected)=4.76, P=0.03 GG=susceptibility to OCOPD; and
4. Allele. G vs C for COPD vs resistant, Odds ratio (OR)=0.5, 95% confidence limits 0.3-0.9, χ² (Yates corrected)=5.65, p=0.02, G=susceptibility to OCOPD.
   Thus, for the −765 C/G promoter polymorphisms of cyclo-oxygenase 2 gene, the C allele and CC/CG genotype was found to be significantly greater in the exposed resistant cohort compared to the OCOPD cohort (OR=2.1, P=0.02 and OR=2.2, P=0.03) consistent with a protective role. This greater frequency compared to the blood donor cohort also suggests that the C allele (CC genotype) is over represented in the resistant group (see Table 1E).

### EXAMPLE 3

### Glutathione S Transferase P1 Ile 105 Val (A/G) Polymorphism Allele and Genotype Frequencies in the Exposed COPD Patients, Exposed Resistant Smokers and Controls

The genotype frequency for the above allele was determined in exposed COPD patients, exposed resistant smokers, and controls. The frequencies are shown in the following table.

TABLE 2
Glutathione S Transferase P1 Ile 105 Val (A/G) Polymorphism Allele
And Genotype Frequencies In The Exposed COPD Patients, Exposed
Resistant Smokers And Controls.

| | Allele* | | Genotype | | |
|---|---|---|---|---|---|
| Frequency | A | G | AA | AG | GG |
| Controls n = 186 | 234 | 138 | 71 (38%) | 92 (50%) | 23 (12%) |
| (%) | (63%) | (37%) | | | |
| Exposed COPD | 159 | 87 | 52 (42%) | 55 (45%) | 16 (13%) |
| n = 123 (%) | (65%) | (36%) | | | |
| Exposed Resistant | 136 | 60 | 44 (45%) | 48 (49%) | 6 (6%) |
| n = 98 (%) | (69%) | (31%) | | | |

*number of chromosomes (2n)

A mathematical analysis of the data in the table indicated that:
1. Genotype. GG vs AG/AA for COPD vs resistant, Odds ratio (OR)=2.3, 95% confidence limits=0.8-6.9, χ² (Yates uncorrected)=2.88, p=0.09, GG genotype=susceptibility to OCOPD.
   Thus, for the Ile 105 Val (A/G) polymorphism of the glutathione S transferase P gene, the GG genotype were found to be greater in the exposed COPD cohort compared to the resistant cohort (OR=2.3, P=0.09 consistent with a susceptibility role. (see Table 2).

### EXAMPLE 4

### Interleukin 18 105 C/A Polymorphism Allele and Genotype Frequency in the Exposed COPD Patients, Exposed Resistant Smokers and Controls

The genotype frequency for the above allele was determined in exposed COPD patients, exposed resistant smokers, and controls. The frequencies are shown in the following table.

TABLE 3a
Interleukin 18 105 C/A Polymorphism Allele And
Genotype Frequency In The Exposed COPD
Patients, Exposed Resistant Smokers And Controls.

| | Allele* | | Genotype | | |
|---|---|---|---|---|---|
| Frequency | C | A | CC | AC | AA |
| Controls n = 185 | 119 | 251 | 22 (12%) | 75 (40%) | 88 (48%) |
| (%) | (32%) | (68%) | | | |
| Exposed COPD | 62 | 182 | 12 (10%) | 38 (31%) | 72¹ (59%) |
| n = 122 (%) | (25%) | (75%) | | | |
| Exposed Resistant | 60 | 136 | 6³ (6%) | 48 (49%) | 44² (45%) |
| n = 98 (%) | (31%) | (69%) | | | |

*number of chromosomes (2n)

A mathematical analysis of the data in the table indicated that:
1. Genotype. AA vs AC/CC for COPD vs resistant, Odds ratio (OR)=1.8, 95% confidence limits=1.0-3.1, χ² (Yates corrected)=3.8, p=0.05, AA=susceptibility to OCOPD, and
2. Genotype. AA vs AC/CC for COPD vs controls, Odds ratio (OR)=1.6, 95% confidence limits 1.0-2.6, χ² (Yates uncorrected)=3.86, p=0.05 AA=susceptibility to OCOPD.
   Thus, for the 105 C/A polymorphism of the interleukin-18 gene, the AA genotype was found to be significantly greater in the exposed COPD cohort compared to the resistant cohort (OR=1.8, P=0.05) consistent with a susceptibility role. The AA genotype was also greater in the exposed COPD cohort compared with controls (OR 1.6, P=0.05) consistent with a susceptibility role (see Table 3a).

### EXAMPLE 5

### Interleukin 18-133 G/C Polymorphism Allele and Genotype Frequencies in the Exposed COPD Patients, Exposed Resistant Smokers And Controls

The genotype frequency for the above allele was determined in exposed COPD patients, exposed resistant smokers, and controls. The frequencies are shown in the following table.

TABLE 3b
Interleukin 18 - 133 G/C Polymorphism Allele
And Genotype Frequencies In The Exposed COPD
Patients, Exposed Resistant Smokers And Controls.

| | Allele* | | Genotype | | |
|---|---|---|---|---|---|
| Frequency | G | C | GG | GC | CC |
| Controls n = 188 | 121 (32%) | 255 | 23 | 75 (40%) | 90 (48%) |
| (%) | | (68%) | (12%) | | |
| Exposed COPD | 62 (25%) | 182 | 12 | 38 (31%) | 72¹ (59%) |
| n = 122 | | (75%) | (10%) | | |
| Exposed Resistant | 60 (31%) | 134 | 6³ | 48 (50%) | 43² (44%) |
| n = 97 (%) | | (69%) | (6%) | | |

*number of chromosomes (2n)

A mathematical analysis of the data in the table indicated that:
1. Genotype. CC vs CG/GG for COPD vs controls, Odds ratio (OR)=1.6, 95% confidence limits=1.0-2.6, χ² (Yates uncorrected)=3.68, p=0.05 CC=susceptibility to OCOPD; and
2. Genotype. CC vs CG/GG for COPD vs resistant, Odds ratio (OR)=1.8, 95% confidence limits 1.0-3.2, χ² (Yates corrected)=4.10, p=0.04 CC=susceptibility to OCOPD.
   Thus, for the −133 G/C promoter polymorphism of the interleukin-18 gene, the CC genotype were found to be significantly greater in the exposed COPD cohort compared to the controls (OR=1.6, P=0.05) consistent with a susceptibility role. The CC genotype was also greater in the exposed COPD cohort compared with resistant smokers (OR=1.8, P=0.04) consistent with a susceptibility role (see Table 3b).

### EXAMPLE 6

### Interleukin 8-251 A/T Polymorphism Allele and Genotype Frequencies in the Exposed COPD Patients, Exposed Resistant Smokers And Controls

The genotype frequency for the above allele was determined in exposed COPD patients, exposed resistant smokers, and controls. The frequencies are shown in the following table.

TABLE 4
Interleukin 8 - 251 A/T Polymorphism Allele And
Genotype Frequencies In The Exposed COPD Patients,
Exposed Resistant Smokers And Controls.

| | Allele* | | Genotype | | |
|---|---|---|---|---|---|
| Frequency | A | T | AA | AT | TT |
| Controls n = 188 | 175 | 201 | 39 (21%) | 97 (52%) | 52 (28%) |
| (%) | (47%) | (53%) | | | |
| Exposed COPD | 101 | 131 | 21 (18%) | 59 (51%) | 36¹ (31%) |
| n = 116 | (44%) | (56%) | | | |
| Exposed Resistant | 94 | 92 | 26³ (28%) | 42 (45%) | 25² (27%) |
| n = 93 (%) | (50%) | (49%) | | | |

*number of chromosomes (2n)

A mathematical analysis of the data in the table indicated that:
1. Genotype. AA vs AT/TT for COPD vs resistant, Odds ratio (OR)=1.8, 95% confidence limits=0.9-3.6, χ² (Yates uncorrected)=2.88, p=0.09,
AA=protective for OCOPD; and
2. Allele. A vs T for COPD vs resistant, Odds ratio (OR)=1.3, 95% confidence limits=0.9-2.0, χ² (Yates uncorrected)=2.3, p=0.15
A=protective for OCOPD.
   Thus, for the −251 A/T polymorphism of Interleukin-8, the A allele and AA genotype was found to be greater in the exposed COPD cohort compared to resistant smokers (OR=1.3, P=0.15 and OR=1.8, P=0.09) a trend consistent with a protective role (Table 4).

### EXAMPLE 7

### Vitamin D Binding Protein Lys 420 Thr (A/C) Polymorphism Allele and Genotype Frequencies in the Exposed COPD Patients, Exposed Resistant Smokers and Controls

The genotype frequency for the above allele was determined in exposed COPD patients, exposed resistant smokers, and controls. The frequencies are shown in the following table.

TABLE 5a
Vitamin D Binding Protein Lys 420 Thr (A/C) Polymorphism
Allele And Genotype Frequencies In The Exposed
Copd Patients, Exposed Resistant Smokers And Controls.

| | Allele* | | Genotype | | |
|---|---|---|---|---|---|
| Frequency | A | C | AA | AC | CC |
| Controls n = | 113 | 265 | 17 (9%) | 79 (42%) | 93 (49%) |
| 189 (%) | (30%) | (70%) | | | |
| Exposed COPD | 62 | 182 | 5 (4%) | 52 (43%) | 65 (53%) |
| n = 122 (%) | (25%) | (75%) | | | |
| Exposed Resistant | 73 | 125 | 12 (12%) | 49 (50%) | 38 (38%) |
| n = 99 (%) | (37%) | (63%) | | | |

*number of chromosomes (2n)

A mathematical analysis of the data in the table indicated that:
1. Genotype. AA vs AC/CC for resistant vs COPD, Odds ratio (OR)=3.2, 95% confidence limits=1.0-11.0, χ² (Yates corrected)=3.89, p=0.05,
AA genotype =protective for OCOPD;
2. Allele. A vs C for resistant vs COPD, Odds ratio (OR)=1.7, 95% confidence limits 1.1-2.6, χ² (Yates corrected)=6.24, p=0.01
A allele=protective for OCOPD; and
3. Genotype. CC vs AC/AA for COPD vs resistant, Odds ratio (OR)=1.8, 95% confidence limits=1.0-3.3, χ² (Yates corrected)=4.29, p=0.04,
CC genotype=susceptibility to OCOPD.
   Thus, for of the Lys 420 Thr (A/C) polymorphism of the Vitamin D binding protein gene, the A allele and AA genotype were found to be greater in the exposed resistant smoker cohort compared to the COPD cohort (OR=1.7, P=0.01 and OR=3.2, P=0.05 respectively) consistent with a protective role. (see Table 5a). Conversely, the CC genotype was found to be greater in the exposed COPD cohort compared to the resistant cohort (OR=1.8, P=0.04) consistent with a susceptibility role (Table 5a).

### EXAMPLE 8

### Vitamin D Binding Protein Glu 416 Asp (T/G) Polymorphism Allele and Genotype Frequencies in the Exposed COPD Patients, Exposed Resistant Smokers And Controls

The genotype frequency for the above allele was determined in exposed COPD patients, exposed resistant smokers, and controls. The frequencies are shown in the following table.

TABLE 5b
Vitamin D Binding Protein Glu 416 Asp (T/G) Polymorphism
Allele And Genotype Frequencies In The Exposed COPD Patients,
Exposed Resistant Smokers And Controls.

| | Allele* | | Genotype | | |
|---|---|---|---|---|---|
| Frequency | T | G | TT | TG | GG |
| Controls n = 189 | 163 | 215 | 35 (19%) | 93 (49%) | 61 (32%) |
| (%) | (43%) | (57%) | | | |
| Exposed COPD | 109 | 135 | 25 (21%) | 59 (48%) | 38 (31%) |
| n = 122 (%) | (45%) | (55%) | | | |
| Exposed Resistant | 103 | 95 | 23 (23%) | 57 (58%) | 19 (19%) |
| n = 99 (%) | (52%) | (48%) | | | |

*number of chromosomes (2n)

A mathematical analysis of the data in the table indicated that:
1. Genotype. TT/TG vs GG for resistant vs COPD, Odds ratio (OR)=1.9, 95% confidence limits=1.0-38, χ² (Yates uncorrected)=4.08, p=0.04,
TT/TG genotype=protective for OCOPD;
2. Allele. T vs G for resistant vs COPD, Odds ratio (OR)=1.3, 95% confidence limits 0.9-2.0, χ² (Yates uncorrected)=2.36, p=0.12
A allele=protective for OCOPD; and
3. Genotype. GG vs TT/TG for COPD vs resistant, Odds ratio (OR)=0.5, 95% confidence limits=0.3-1.0, χ² (Yates uncorrected)=4.1, p=0.04,
GG genotype=susceptibility to OCOPD.
   Thus, for the Glu 416 Asp (T/G) polymorphism of the Vitamin D binding protein gene, the T allele and TT/TG genotype were found to be greater in the exposed resistant smoker cohort compared to the COPD cohort (OR=1.3, P=0.12 and OR=1.9, P=0.04 respectively) consistent with a protective role. (see Table 5b). Conversely, the GG genotype was found to be greater in the exposed COPD cohort compared to the resistant cohort (OR=0.5, P=0.04) consistent with a susceptibility role (Table 5b).

### EXAMPLE 9

### Microsomal Epxoide Hydrolase R/R Exon 3 T/C Polymorphism Allele and Enotype Frequencies in the Exposed COPD Patients, Exposed Resistant Smokers and Controls

The genotype frequency for the above allele was determined in exposed COPD patients, exposed resistant smokers, and controls. The frequencies are shown in the following table.

TABLE 6
Microsomal epxoide hydrolase R/r Exon 3 T/C Polymorphism Allele
And Genotype Frequencies In The Exposed COPD Patients,
Exposed Resistant Smokers And Controls.

| | Allele* | | Genotype | | |
|---|---|---|---|---|---|
| Frequency | r | R | rr | Rr | RR |
| Controls n = 184 | 228 | 140 | 77 | 74 (40%) | 33 (18%) |
| (%) | (62%) | (38%) | (42%) | | |
| Exposed COPD | 144 | 52 | 55 | 34 (35%) | 9 (9%) |
| n = 98 (%) | (74%) | (26%) | (56%) | | |
| Exposed Resistant | 135 | 69 | 52 | 31 (30%) | 19 (19%) |
| n = 102 (%) | (66%) | (34%) | (51%) | | |

*number of chromosomes (2n)

A mathematical analysis of the data in the table indicated that:
1. Genotype. RR vs Rr/rr for resistant vs COPD, Odds ratio (OR)=2.3, 95% confidence limits=0.9-5.8, χ² (Yates uncorrected)=3.7, p=0.05, RR genotype=protective for OCOPD.
   Thus, for the exon 3 T/C (R/r) polymorphism of the microsomal epoxide hydrolase gene, the RR genotype was found to be greater in the exposed resistant cohort compared to the COPD (OR=2.3, P=0.05) consistent with a protective role (Table 6).

### EXAMPLE 10

### Super Oxide Dismutase 3 Arg 312 Gln Polymorphism Allele and Genotype Frequencies in the Exposed COPD Patients, Exposed Resistant Smokers and Controls

The genotype frequency for the above allele was determined in exposed COPD patients, exposed resistant smokers, and controls. The frequencies are shown in the following table.

TABLE 7
Super oxide dismutase 3 Arg 312 Gln Polymorphism Allele And
Genotype Frequencies In The Exposed COPD Patients,
Exposed Resistant Smokers And Controls.

| | Allele* | | Genotype | | |
|---|---|---|---|---|---|
| Frequency | A | G | AA | AG | GG |
| Controls n = 190 | 371 | 9 | 183 (96%) | 5 (3%) | 2 (1%) |
| (%) | (98%) | (2%) | | | |
| Exposed COPD | 199 | 1 (1%) | 99 (99%) | 1 (1%) | 0 (0%) |
| n = 100 (%) | (99%) | | | | |
| Exposed Resistant | 193 | 11 | 92 (90%) | 9 (9%) | 1 (1%) |
| n = 102 (%) | (95%) | (5%) | | | |

*number of chromosomes (2n)

A mathematical analysis of the data in the table indicated that:
1. Genotype. AG/GG vs AA for resistant vs COPD, Odds ratio (OR)=10.8, 95% confidence limits=1.4-229, χ² (Yates corrected)=5.99 p=0.01,
AG/GG genotype=protective for OCOPD
AA=susceptibility to OCOPD; and
2. Allele. G vs A for resistant vs COPD, Odds ratio (OR)=11.3, 95% confidence limits 1.5-237, χ² (Yates corrected)=6.77, p=0.001
G allele=protective for OCOPD
A allele=susceptibility to OCOPD.
   Thus, for the Arg 312 Gln (AC) polymorphism of the superoxide dismutase 3 gene, the G allele and AG/GG (AC/CC) genotype was found to be greater in the exposed resistant cohort compared to the COPD cohort (OR=11.3, P=0.001 and OR=10.8, P=0.01) consistent with a protective role (while the A allele and AA genotype are susceptible) (Table 7).

### EXAMPLE 11

### α1-Antitrypsin S Polymorphism Allele and Genotype Frequencies in the Exposed COPD Patients and Exposed Resistant Smokers

The genotype frequency for the above allele was determined in exposed COPD patients and exposed resistant smokers. The frequencies are shown in the following table.

TABLE 8
α1-Antitrypsin S Polymorphism Allele And Genotype Frequencies
In The Exposed COPD Patients And Exposed Resistant Smokers.

| | Allele* | | Genotype | | |
|---|---|---|---|---|---|
| Frequency | M | S | MM | MS | SS |
| Exposed COPD | 171 | 5 | 83 (94%) | 5 (6%) | 0 (0%) |
| n = 88 (%) | (97%) | (3%) | | | |
| Exposed | 175 | 13 | 81 (86%) | 13 (14%) | 0 (0%) |
| Resistant n = 94 | (93%) | (7%) | | | |
| (%) | | | | | |

*number of chromosomes (2n)

A mathematical analysis of the data in the table indicated that:
1. Genotype. MS vs MM for Resistant vs COPD, Odds ratio (OR)=2.7, 95% confidence limits 0.8-9.0, χ² (Yates uncorrected)=3.4, p=0.07,
MS=protective for OCOPD; and
2. Allele: S vs M allele for resistant vs COPD, Odds ratio (OR)=2.5, 95% confidence limits 0.8-8.4, χ² (Yates uncorrected)=3.24, p=0.07
S=protective for OCOPD. Thus, for the α1-antitrypsin S polymorphism, the S allele and MS/SS genotype was found to be greater in the resistant smokers compared to COPD cohort (OR=2.5, P=0.07 and OR=2.7, P=0.07) consistent with a protective role (Table 8).

### EXAMPLE 12

### α1-Antitrypsin 3′ 1237 G/A (T/t) Polymorphism Allele and Genotype Frequencies in the Exposed COPD Patients and Exposed Resistant Smokers

The genotype frequency for the above allele was determined in exposed COPD patients, exposed resistant smokers, and controls. The frequencies are shown in the following table.

TABLE 9
α1-antitrypsin 3′ 1237 G/A (T/t) Polymorphism Allele
And Genotype Frequencies In The Exposed COPD
Patients And Exposed Resistant Smokers.

| | Allele* | | Genotype | | |
|---|---|---|---|---|---|
| Frequency | T | t | TT | Tt | tt |
| Controls n = 178 | 345 | 11 (3%) | 167 (94%) | 11 (6%) | 0 (0%) |
| (%) | (97%) | | | | |
| Exposed COPD | 109 | 13 (11%) | 50 (82%) | 9 (15%) | 2 (3%) |
| n = 61 (%) | (89%) | | | | |
| Exposed Resistant | 67 | 3 (4%) | 32 (91%) | 3 (9%) | 0 (0%) |
| n = 35 (%) | (96%) | | | | |

*number of chromosomes (2n)

A mathematical analysis of the data in the table indicated that:
1. Genotype: Tt/tt vs TT for COPD vs controls, Odd's Ratio (OR)=3.34, 95% confidence limits 1.3-8.9, χ² (Yates corrected)=6.28, p=0.01.
Tt/tt=susceptibility to OCOPD; and
2. Allele: t vs T for COPD vs controls, Odd's Ratio (OR)=2.5, 95% confidence limits 1.0-6.3, χ² (Yates corrected)=4.1, p=0.04.
t=susceptibility to OCOPD.
   Thus, for the 3′ 1237 G/A (T/t) polymorphism of the α1-antitrypsin gene, the t allele and Tt/tt genotype was found to be significantly greater in the exposed COPD cohort compared to controls (OR=2.5, P=0.04 and OR=3.3, P=0.01) consistent with a susceptibility role (Table 9).

### EXAMPLE 13

### Toll-Like Receptor 4 Asp 299 Gly A/G Polymorphism Allele and Genotype Frequencies in the Exposed COPD Patients and Exposed Resistant Smokers

The genotype frequency for the above allele was determined in exposed COPD patients and exposed resistant smokers. The frequencies are shown in the following table.

TABLE 10
Toll-Like Receptor 4 Asp 299 Gly A/G Polymorphism Allele And
Genotype Frequencies In The Exposed COPD Patients
And Exposed Resistant Smokers.

| | Allele* | | Genotype | | |
|---|---|---|---|---|---|
| Frequency | A | G | AA | AG | GG |
| Exposed COPD | 117 | 1 | 58 (98%) | 1 (2%) | 0 (0%) |
| n = 60 (%) | (98%) | (2%) | | | |
| Exposed Resistant | 65 | 3 | 31 (91%) | 3 (9%) | 0 (0%) |
| n = 34 (%) | (96%) | (4%) | | | |

*number of chromosomes (2n)

A mathematical analysis of the data in the table indicated that:
1. Genotype AG vs AA in resistant vs COPD, Odd's Ratio (OR)=5.61, 95% confidence limits 0.5-146, χ² (Yates uncorrected)=2.66, p=0.10, AG=protective for OCOPD.
   Thus, for the Asp 299 Gly A/G polymorphism of the toll-like receptor 4 gene, the AG(GG) genotype was found to be greater in the exposed resistant cohort compared to COPD (OR=5.6, P=0.10) consistent with a protective role Table 10).

### EXAMPLE 14

### Beta2-Adrenoreceptor Gln 27 Glu Polymorphism Allele and Genotype Frequency in the Exposed COPD Patients, Exposed Resistant Smokers and Controls

The genotype frequency for the above allele was determined in exposed COPD patients, exposed resistant smokers, and controls. The frequencies are shown in the following table.

TABLE 11
Beta2-Adrenoreceptor Gln 27 Glu Polymorphism Allele
And Genotype Frequency In The Exposed COPD Patients,
Exposed Resistant Smokers And Controls.

| | Allele* | | Genotype | | |
|---|---|---|---|---|---|
| Frequency | C | G | CC | CG | GG |
| Controls n = 186 | 204 | 168 | 57 (31%) | 90 (48%) | 39 (21%) |
| (%) | (55%) | (45%) | | | |
| Exposed COPD | 129 | 115 | 32 (26%) | 65 (53%) | 25¹ (21%) |
| n = 122 (%) | (53%) | (47%) | | | |
| Exposed Resistant | 117 | 81 | 38 (38%) | 41 (41%) | 20² (20%) |
| n = 99 (%) | (59%) | (41%) | | | |

*number of chromosomes (2n)

A mathematical analysis of the data in the table indicated that:
1. Genotype. CC vs CG/GG for resistant vs COPD, Odds ratio (OR)=1.75, 95% confidence limits=1.0-3.2, χ² (Yates uncorrected)=3.73, p=0.05,
CC=protective for OCOPD.
   Thus, for the Gln27Glu polymorphism of the β2 adrenergic receptor gene, the CC genotype was found to be significantly greater in the exposed resistant cohort compared to the COPD cohort (OR=1.75, P=0.05) suggesting a possible protective role to aero-pollutants associated with this genotype. (see Table 11).

### EXAMPLE 15

### Interleukin 11 (IL-11)-518 G/A Polymorphism Allele and Genotype Frequencies in the Exposed COPD Patients and Exposed Resistant Smokers

The genotype frequency for the above allele was determined in exposed COPD patients and exposed resistant smokers. The frequencies are shown in the following table.

TABLE 12
Interleukin 11 (IL-11)-518 G/A Polymorphism Allele
And Genotype Frequencies In The Exposed COPD
Patients And Exposed Resistant Smokers.

| | Allele* | | Genotype | | |
|---|---|---|---|---|---|
| Frequency | A | G | AA | AG | GG |
| Exposed COPD | 110 | 128 | 22 | 66 (55%) | 31 (26%) |
| n = 119 (%) | (46%) | (54%) | (19%) | | |
| Exposed Resistant | 103 | 93 | 26 | 51 (52%) | 21 (21%) |
| n = 98 (%) | (53%) | (47%) | (27%) | | |

*number of chromosomes (2n)

A mathematical analysis of the data in the table indicated that:
1. Genotype: AA vs AG/GG for resistant vs COPD, Odd's Ratio (OR)=1.6, 95% confidence limits 0.8-32, χ² (Yates uncorrected)=2.02, p=0.16
AA=protective for OCOPD.
   Thus, for the −518 G/A polymorphism of the interleukin −8 gene, the AA genotype was greater in the exposed resistant cohort compared with the COPD (OR=1.6, P=0.16) consistent with a protective role (Table 12).

### EXAMPLE 16

### Interleukin-13 −1055 C/T Promoter Polymorphism Allele and Genotype Frequencies in the Exposed COPD Patients, Exposed Resistant Smokers and Controls

The genotype frequency for the above allele was determined in exposed COPD patients, exposed resistant smokers, and controls. The frequencies are shown in the following table.

TABLE 13
Interleukin-13 - 1055 C/T Promoter Polymorphism Allele
And Genotype Frequencies In The Exposed COPD Patients,
Exposed Resistant Smokers And Controls.

| | Allele* | | Genotype | | |
|---|---|---|---|---|---|
| Frequency | T | C | TT | TC | CC |
| Controls n = 182 | 65 (18%) | 299 | 5 (3%) | 55 (30%) | 122 (67%) |
| (%) | | (82%) | | | |
| Exposed COPD | 53 (22%) | 189 | 5 (4%) | 43 (36%) | 73 (60%) |
| n = 121 (%) | | (78%) | | | |
| Exposed Resistant | 31 (16%) | 163 | 1 (1%) | 29 (30%) | 67 (69%) |
| n = 97 (%) | | (84%) | | | |

*number of chromosomes (2n)

A mathematical analysis of the data in the table indicated that:
1. Genotype. TT vs TC/CC for COPD vs resistant, Odds ratio (OR)=6.03, 95% confidence limits 1.1-42, χ² (Yates corrected)=4.9, p=0.03,
TT=susceptibility to OCOPD.
   Thus, for the −1055 (C/T) polymorphism of the interleukin-13 gene, the TT genotype was found to be greater in the exposed COPD cohort compared to the resistant cohort (OR=6.03, P=0.03) consistent with a susceptibility role. (see Table 13).

### EXAMPLE 17

### Plasminogen Activator Inhibitor 1-675 4G/5G Promoter Polymorphism Allele and Genotype Frequencies in the Exposed COPD Patients, Exposed Resistant Smokers and Controls

The genotype frequency for the above allele was determined in exposed COPD patients, exposed resistant smokers, and controls. The frequencies are shown in the following table.

TABLE 14
Plasminogen Activator Inhibitor 1 - 675 4G/5G Promoter
Polymorphism Allele And Genotype Frequencies In The Exposed
COPD Patients, Exposed Resistant Smokers And Controls.

| | Allele* | | Genotype | | |
|---|---|---|---|---|---|
| Frequency | 5G | 4G | 5G5G | 5G4G | 4G4G |
| Controls n = 186 | 158 | 214 | 31 | 96 (52%) | 59 |
| (%) | (42%) | (58%) | (17%) | | (32%) |
| Exposed COPD | 115³ | 129 | 29^{1,2} | 57 (47%) | 36 |
| n = 122 (%) | (47%) | (53%) | (24%) | | (30%) |
| Exposed | 76 | 120 | 14 | 48 (49%) | 36^{1,2} |
| Resistant n = 98 | (39%) | (61%) | (14%) | | (37%) |
| (%) | | | | | |

*number of chromosomes (2n)

A mathematical analysis of the data in the table indicated that:
1. Genotype. 5G5G vs rest for COPD vs resistant, Odds ratio (OR)=1.9, 95% confidence limits 0.9-4.0, χ² (Yates uncorrected)=3.11, p=0.08,
5G5G=susceptibile to OCOPD; and
2. Allele. 5G vs 4G for COPD vs resistant, Odds ratio (OR)=1.4, 95% confidence limits 0.9-2.1, χ² (Yates corrected)=3.1, p=0.08
5G=susceptibile to OCOPD.
   Thus, for the −675 4G/5G promoter polymorphism of the plasminogen activator inhibitor gene, the 5G allele and 5G5G genotype was found to be significantly greater in the exposed COPD cohort compared to the resistant smoker cohort (OR=1.4, P=0.08 and OR=1.9, P=0.08) consistent with a susceptibility role. The greater frequency of the 5G5G in exposed COPD compared to the blood donor cohort also suggests that the 5G5G genotype is associated with susceptibility (see Table 14).

### EXAMPLE 18

### Nitric Oxide Synthase 3 Asp 298 Glu (T/G) Polymorphism Allele and Genotype Frequencies in the Exposed COPD Patients, Exposed Resistant Smokers and Controls

The genotype frequency for the above allele was determined in exposed COPD patients, exposed resistant smokers, and controls. The frequencies are shown in the following table.

TABLE 15
Nitric Oxide Synthase 3 Asp 298 Glu (T/G) Polymorphism Allele
And Genotype Frequencies In The Exposed COPD Patients,
Exposed Resistant Smokers And Controls.

| | Allele* | | Genotype | | |
|---|---|---|---|---|---|
| Frequency | T | G | TT | TG | GG |
| Controls n = | 108 (30%) | 258 | 13 (7%) | 82 (45%) | 88 (48%) |
| 183 (%) | | (70%) | | | |
| Exposed COPD | 71 (30%) | 169 | 10 (8%) | 51 (43%) | 59 (49%) |
| n = 120 (%) | | (70%) | | | |
| Exposed | 71 (36%) | 127 | 15¹ (15%) | 41 (41%) | 43 (43%) |
| Resistant | | (64%) | | | |
| n = 99 (%) | | | | | |

*number of chromosomes (2n)

A mathematical analysis of the data in the table indicated that:
1. Genotype. TT vs TG/GG for resistant vs controls, Odds ratio (OR)=2.3, 95% confidence limits 1.0-5.5, χ² (Yates corrected)=3.80, p=0.05,
TT genotype=protective for OCOPD; and
2. Genotype. TT vs TG/GG for resistant vs COPD, Odds ratio (OR)=1.9, 95% confidence limits 0.8-5.0, χ² (Yates uncorrected)=2.49, p=0.11,
TT genotype=protective for OCOPD.
   Thus, for the 298 Asp/Glu (T/G) polymorphism of the nitric oxide synthase (NOS3) gene, the TT genotype was found to be significantly greater in the resistant smoker cohort compared to the blood donor cohort and COPD cohort (OR=2.3, P=0.05 and OR=1.9, P=0.11) consistent with a protective role. (see Table 15).

### EXAMPLE 19

### Matrix Metalloproteinase 1 (MMP1)-1607 1G/2G Polymorphism Allele and Genotype Frequencies in the Exposed COPD Patients, Exposed Resistant Smokers and Controls

The genotype frequency for the above allele was determined in exposed COPD patients, exposed resistant smokers, and controls. The frequencies are shown in the following table.

TABLE 16
Matrix Metalloproteinase 1 (MMP1) - 1607 1G/2G Polymorphism
Allele And Genotype Frequencies In The Exposed COPD Patients,
Exposed Resistant Smokers And Controls.

| | Allele* | | Genotype | | |
|---|---|---|---|---|---|
| Frequency | 1G | 2G | 1G1G | 1G2G | 2G2G |
| Controls n = | 214 (61%) | 134 (39%) | 68 (39%) | 78 (45%) | 28 (16%) |
| 174 (%) | | | | | |
| Exposed | 90 (48%) | 96 (52%) | 24 (26%) | 42 (45%) | 27 (29%) |
| COPD | | | | | |
| n = 93 (%) | | | | | |
| Exposed | 99 (53%) | 89 (47%) | 25 (27%) | 49 (52%) | 20 (21%) |
| Resistant | | | | | |
| n = 94 (%) | | | | | |

*number of chromosomes (2n)

A mathematical analysis of the data in the table indicated that:
1. Genotype. 2G2G vs 1G1G/1G2G for COPD vs controls, Odds ratio (OR)=2.1, 95% confidence limits 1.1-4.1, χ² (Yates corrected)=5.44, p=0.02,
2G2G genotype=susceptibility for OCOPD and
2. Allele. 2G vs 1G for COPD vs controls, Odds ratio (OR)=1.7, 95% confidence limits 1.2-2.5, χ² (Yates corrected)=7.97, p=0.005,
2G=susceptibility for OCOPD.
   Thus, for the −1607 1G/2G polymorphism of the matrix metalloproteinase 1 gene, the 2G allele and 2G2G genotype were found to be significantly greater in the exposed COPD cohort compared to the blood donor cohort (OR=1.7, P=0.005 and OR=2.1, P=0.02) consistent with a susceptibility role. (see Table 16).

Table 17 summarizes the above results and examples.

TABLE 17
Summary Table Of Protective And Susceptibility
Polymorphisms In Occupational COPD

| Gene | Polymorphism | Role |
|---|---|---|
| Cyclo-oxygenase (Cox) 2 | Cox 2-765 G/C | CC/CG protective |
| | | GG susceptibility |
| β2-adrenoreceptor (ADRB2) | ADRB2 Gln 27 Glu | CC protective |
| Interleukin-18 (IL-18) | IL-18-133 C/G | CC susceptibility |
| Interleukin-18 (IL-18) | IL-18 105 A/C | AA susceptibility |
| Plasminogen activator inhibitor 1 (PAI-1) | PAI-1-675 4G/5G | 5G5G susceptibility |
| Nitric Oxide synthase 3 (NOS3) | NOS3 298 Asp/Glu | TT protective |
| Vitamin D Binding Protein (VDBR) | VDBR Lys 420 Thr | AA protective |
| | | CC susceptibility |
| Vitamin D Binding Protein (VDBR) | VDBP Glu 416 Asp | TT/TG protective |
| | | GG susceptibility |
| Glutathione S Transferase (GSTP1) | GSTP1 Ile 105 Val | GG susceptibility |
| Superoxide dismutase 3 (SOD3) | SOD3 Arg 312 Gln | AG/GG protective |
| | | AA susceptibility |
| α1-antitrypsin (α1AT) | α1AT 3′ 1237 G/A (T/t) | Tt/tt susceptibility |
| α1-antitrypsin (α1AT) | α1AT S allele | MS protective |
| Toll-like receptor 4 (TLR4) | TLR4 Asp 299 Gly A/G | AG/GG protective |
| Interleukin-8 (IL-8) | IL-8-251 A/T | AA protective |
| Interleukin 11 (IL-11) | IL-11-518 G/A | AA protective |
| Microsomal epoxide hydrolase (MEH) | MEH Exon 3 T/C (r/R) | RR protective |
| Interleukin 13 (IL-13) | IL-13-1055 C/T | TT susceptibility |
| Matrix Metalloproteinase 1 (MMP1) | MMP1-1607 1G/2G | 2G2G susceptibility |

### EXAMPLE 20

### Combined Frequencies of the Presence or Absence of Protective Genotypes (Cox 2-765 CC/CG, NOS3 298 TT, A1at MS/SS, SOD3 AG/GG, MEH Exon 3 RR, VDBR 420 AA) in the Exposed Smoking Subjects (OCOPD Subjects and Resistant Smokers)

In addition to examining the individual frequencies, the frequencies of the presence or absence of protective genotypes in various combinations were also examined. The frequency of having impaired lung function (COPD) according to the presence or absence of 6 of the protective polymorphisms (Cox 2 −765 CC/CG, NOS3 298 TT, α1AT MS/SS, SOD3 AG/GG, MEH Exon 3 RR, VDBR 420 AA) was also examined. The results are summarized in Table 18.

TABLE 18
Combined Frequencies Of The Presence Or Absence Of Protective
Genotypes (Cox 2-765 CC/CG, NOS3 298 TT,
α1AT MS/SS, SOD3 AG/GG, MEH
Exon 3 RR, VDBR 420 AA) In The Exposed Smoking Subjects
(OCOPD Subjects And Resistant Smokers).

| | Number of protective polymorphisms | | | |
|---|---|---|---|---|
| Cohorts | 0 | 1 | ≧2 | Total |
| Exposed COPD | 34 (27%) | 51 (41%) | 39 (32%) | 124 |
| Exposed | 20 (19%) | 31 (30%) | 53 (51%) | 104 |
| Resistant | | | | |
| smokers | | | | |
| % of smokers | 34/54 (63%) | 51/82 (62%) | 39/92 (42%) | |
| with COPD | | | | |

| | Odd's | | | |
|---|---|---|---|---|
| Comparison | ratio | 95% CI | χ² | P value |
| 0 vs 1 vs 2+, Exposed Resist vs | — | — | 16.2 | 0.003 |
| Exposed COPD | | | | |
| 2+ vs 0-1, Exposed Resist vs | 2.3 | 1.3-4.0 | 8.15 | 0.004 |
| Exposed COPD | | | | |
| 0 vs 2+, Exposed COPD vs | 2.3 | 1.1-4.9 | 4.97 | 0.03 |
| Exposed Resist | | | | |

In Table 18 it can be seen that for those with 0 protective polymorphisms, 63% were COPD in comparison with those with 2+ protective polymorphisms where it was only 42% with Odd's ratio of about 2.

### EXAMPLE 21

### Combined Frequencies of the Presence or Absence of Susceptibility Genotypes (MMP1-1607 2G2G, GSTP1 105 GG, PAI-1-675 5G5G, IL-13 −1055 TT, VDBP 416 GG) in the Exposed Smoking Subjects (OCOPD Subjects and Resistant Smokers)

In addition to examining the individual frequencies, the frequencies of the presence or absence of susceptibility genotypes in various combinations were also examined. In the subjects exposed to aero-pollutants (both COPD and resistant) the frequency of having impaired lung function (COPD) according to the presence or absence of 5 of the susceptibility polymorphisms (MMP1 −1607 2G2G, GSTP1 105 GG, PAI-1 −675 5G5G, IL-13 −1055 TT, VDBP 416 GG) was also examined. The results are shown in Table 19.

TABLE 19
Combined Frequencies Of The Presence Or Absence Of
Susceptibility Genotypes (MMP1-1607 2G2G,
GSTP1 105 GG, PAI-1-675 5G5G,
IL-13-1055 TT, VDBP 416 GG) In The Exposed Smoking Subjects
(OCOPD Subjects And Resistant Smokers).

| | Number of susceptibility polymorphisms | | | |
|---|---|---|---|---|
| Cohorts | 0 | 1 | ≧2 | Total |
| Exposed | 45 (36%) | 55 (44%) | 24 (20%) | 124 |
| COPD | | | | |
| Exposed | 55 (54%) | 37 (37%) | 9 (9%) | 101 |
| Resistant | | | | |
| smokers | | | | |
| % of | 45/100 (45%) | 55/92 (60%) | 24/33 (73%) | |
| smokers | | | | |
| with COPD | | | | |

| Comparison | Odd's ratio | 95% CI | χ2 | P value |
|---|---|---|---|---|
| 0 vs 1 vs 2+, Exposed COPD vs | — | — | 9.1 | 0.01 |
| Exposed Resist | | | | |
| 2+ vs 0-1, Exposed COPD vs | 2.5 | 1.0-6.0 | 4.05 | 0.04 |
| Exposed Resist | | | | |
| 0+ vs 1-2+, Exposed Resist vs | 2.1 | 1.2-3.7 | 6.72 | 0.01 |
| Exposed COPD | | | | |

In Table 19 it can be seen that for those with 2+ susceptibility polymorphisms 73% had COPD in comparison with those with 0 susceptibility polymorphisms, in which only 45% had COPD with Odd's ratio of about 2. Thus, there is clearly a correlation between the number of susceptibility polymorphisms and the likelihood that someone will have COPD.

### EXAMPLE 22

### Combined Presence or Absence of Protective and Susceptibility Polymorphisms

In addition to the above examples, the relevance of the combined presence or absence of protective and susceptibility polymorphisms was also examined. Protective polymorphisms were scored as a +1 while susceptibility polymorphisms were scored as a −1. The results are presented in Table 20 below.

TABLE 20
Combined Presence Or Absence Of Protective And Susceptibility
Polymorphisms (Scored As +1 Or −1 Respectively)
In Each Subject Exposed To Aero-Pollutants (Combined
OCOPD And Resistant Exposed Smokers)

| | Score combining protective | | | | | |
|---|---|---|---|---|---|---|
| | and susceptibility polymorphisms | | | | | |
| | −2 | −1 | 0 | 1 | 2 | 3 |
| Exposed COPD | 8 | 28 | 33 | 39 | 11 | 5 |
| n = 124 | | | | | | |
| Exposed | 2 | 11 | 23 | 27 | 23 | 15 |
| Resistant n = 101 | | | | | | |
| % COPD | 80% | 72% | 59% | 59% | 32% | 25% |

Again, the data demonstrate that there is a continuous correlation between the percent of subjects with COPD and the number of susceptibility polymorphisms that the subjects have, as well as a continuing decrease in risk for the presence of additional protective polymorphisms.

The above examples and results show that several polymorphisms were associated with either increased or decreased risk of developing obstructive lung disease in those exposed to work place aero-pollutants and chronic smoking. The associations of individual polymorphisms on their own can be of discriminatory value.

In combination these polymorphisms can further distinguish susceptible workers (with OCOPD) from those with comparable work place and smoking exposure who are resistant. The polymorphisms represent both promoter polymorphisms, thought to modify gene expression and hence protein synthesis, and exonic polymorphisms known to alter amino-acid sequence (and likely expression and/or function) in processes known to underlie lung remodelling. The polymorphisms identified here are found in genes encoding proteins central to these processes which include inflammation, matrix remodelling and oxidant stress.

In the comparison of workers exposed to aero-pollutants/smoking with COPD (OCOPD subjects) and matched smokers with comparable workplace/smoking exposure but near normal lung function, several polymorphism were identified as being found in significantly greater or lesser frequency than in the comparator groups (including the blood donor cohort).
In the analysis of the −765 C/G promoter polymorphisms of cyclo-oxygenase 2 gene, the C allele and CC/CG genotype was found to be significantly greater in the exposed resistant cohort compared to the OCOPD cohort (OR=2.1, P=0.02 and OR=2.2, P=0.03) consistent with a protective role. This greater frequency compared to the blood donor cohort also suggests that the C allele (CC genotype) is over represented in the resistant group (see Table 1E).
In the analysis of the Ile 105 Val (A/G) polymorphism of the glutathione S transferase P gene, the GG genotype were found to be greater in the exposed COPD cohort compared to the resistant cohort (OR=2.3, P=0.09 consistent with a susceptibility role. (see Table 2).
In the analysis of the 105 C/A polymorphism of the interleukin-18 gene, the AA genotype was found to be significantly greater in the exposed COPD cohort compared to the resistant cohort (OR=1.8, P=0.05) consistent with a susceptibility role. The AA genotype was also greater in the exposed COPD cohort compared with controls (OR 1.6, P=0.05) consistent with a susceptibility role (see Table 3a).
In the analysis of the −133 G/C promoter polymorphism of the interleukin-18 gene, the CC genotype were found to be significantly greater in the exposed COPD cohort compared to the controls (OR=1.6, P=0.05) consistent with a susceptibility role. The CC genotype was also greater in the exposed COPD cohort compared with resistant smokers (OR=1.8, P=0.04) consistent with a susceptibility role (see Table 3b).
In the analysis of the −251 A/T polymorphism of Interleukin-8, the A allele and AA genotype was found to be greater in the exposed COPD cohort compared to resistant smokers (OR=1.3, P=0.15 and OR=1.8, P=0.09) a trend consistent with a protective role (Table 4).
In the analysis of the Lys 420 Thr (A/C) polymorphism of the Vitamin D binding protein gene, the A allele and AA genotype were found to be greater in the exposed resistant smoker cohort compared to the COPD cohort (OR=1.7, P=0.01 and OR=3.2, P=0.05 respectively) consistent with a protective role. (see Table 5a). Conversely, the CC genotype was found to be greater in the exposed COPD cohort compared to the resistant cohort (OR=1.8, P=0.04) consistent with a susceptibility role (Table 5a).
In the analysis of the Glu 416 Asp (T/G) polymorphism of the Vitamin D binding protein gene, the T allele and TT/TG genotype were found to be greater in the exposed resistant smoker cohort compared to the COPD cohort (OR=1.3, P=0.12 and OR=1.9, P=0.04 respectively) consistent with a protective role. (see Table 5b). Conversely, the GG genotype was found to be greater in the exposed COPD cohort compared to the resistant cohort (OR=0.5, P=0.04) consistent with a susceptibility role (Table 5b).
In the analysis of the exon 3 T/C (R/r) polymorphism of the microsomal epoxide hydrolase gene, the RR genotype was found to be greater in the exposed resistant cohort compared to the COPD (OR=2.3, P=0.05) consistent with a protective role (Table 6).
In the analysis of the Arg 312 Gln (AC) polymorphism of the superoxide dismutase 3 gene, the G allele and AG/GG (AC/CC) genotype was found to be greater in the exposed resistant cohort compared to the COPD cohort (OR=11.3, P=0.001 and OR=10.8, P=0.01) consistent with a protective role (while the A allele and AA genotype are susceptible) (Table 7).
In the analysis of the α1-antitrypsin S polymorphism, the S allele and MS/SS genotype was found to be greater in the resistant smokers compared to COPD cohort (OR=2.5, P=0.07 and OR=2.7, P=0.07) consistent with a protective role (Table 8).
In the analysis of the 3′ 1237 G/A (T/t) polymorphism of the α1-antitrypsin gene, the t allele and Tt/tt genotype was found to be significantly greater in the exposed COPD cohort compared to controls (OR=2.5, P=0.04 and OR=3.3, P=0.01) consistent with a susceptibility role (Table 9).
In the analysis of the Asp 299 Gly A/G polymorphism of the toll-like receptor 4 gene, the AG(GG) genotype was found to be greater in the exposed resistant cohort compared to COPD (OR=5.6, P=0.10) consistent with a protective role Table 10).
In the analysis of the Gln27Glu polymorphism of the β2 adrenergic receptor gene, the CC genotype was found to be significantly greater in the exposed resistant cohort compared to the COPD cohort (OR=1.75, P=0.05) suggesting a possible protective role to aero-pollutants associated with this genotype. (see Table 11).
In the analysis of the −518 G/A polymorphism of the interleukin −8 gene, the AA genotype was greater in the exposed resistant cohort compared with the COPD (OR=1.6, P=0.16) consistent with a protective role (Table 12).
In the analysis of the −1055 (C/T) polymorphism of the interleukin-13 gene, the TT genotype was found to be greater in the exposed COPD cohort compared to the resistant cohort (OR=6.03, P=0.03) consistent with a susceptibility role. (see Table 13).
In the analysis of the −675 4G/5G promoter polymorphism of the plasminogen activator inhibitor gene, the 5G allele and 5G5G genotype was found to be significantly greater in the exposed COPD cohort compared to the resistant smoker cohort (OR=1.4, P=0.08 and OR=1.9, P=0.08) consistent with a susceptibility role. The greater frequency of the 5G5G in exposed COPD compared to the blood donor cohort also suggests that the 5G5G genotype is associated with susceptibility (see Table 14).
In the analysis of the 298 Asp/Glu (T/G) polymorphism of the nitric oxide synthase (NOS3) gene, the TT genotype was found to be significantly greater in the resistant smoker cohort compared to the blood donor cohort and COPD cohort (OR=2.3, P=0.05 and OR=1.9, P=0.11) consistent with a protective role. (see Table 15).
In the analysis of the −1607 1G/2G polymorphism of the matrix metalloproteinase 1 gene, the 2G allele and 2G2G genotype were found to be significantly greater in the exposed COPD cohort compared to the blood donor cohort (OR=1.7, P=0.005 and OR=2.1, P=0.02) consistent with a susceptibility role. (see Table 16).
In the subjects exposed to aero-pollutants (both COPD and resistant) the frequency of having impaired lung function (COPD) according to the presence or absence of 6 of the protective polymorphisms (Cox 2-765 CC/CG, NOS3 298 TT, α1AT MS/SS, SOD3 AG/GG, MEH Exon 3 RR, VDBR 420 AA) was also examined. In Table 18 it can be seen that for those with 0 protective polymorphisms 63% were COPD in comparison with those with 2+protective polymorphisms it was only 42% with Odd's ratio of about 2.
In the subjects exposed to aero-pollutants (both COPD and resistant) the frequency of having impaired lung function (COPD) according to the presence or absence of 5 of the susceptibility polymorphisms (MMP1 −1607 2G2G, GSTP1 105 GG, PAI-1 −675 5G5G, IL-13 −1055 TT, VDBP 416 GG) was also examined. In Table 19 it can be seen that for those with 2+ susceptibility polymorphisms 73% were COPD in comparison with those with 0 susceptibility polymorphisms it was only 45% with Odd's ratio of about 2.

It is accepted that the disposition to chronic obstructive lung diseases (e.g., OCOPD) is the result of the combined effects of the individual's genetic makeup and their lifetime exposure to various aero-pollutants. Similarly it is accepted that OCOPD encompasses several obstructive lung diseases and characterised by impaired expiratory flow rates (eg FEV1). The data herein suggest that several genes can contribute to the development of OCOPD following exposure to work place aero-pollutants. A number of genetic mutations working in combination either promoting or protecting the lungs from damage can be involved in elevated resistance or susceptibility.

From the analyses of the individual polymorphisms, 11 protective genotypes were identified and analyzed for their frequencies in the smoker cohort consisting of resistant smokers and those with OCOPD. When the frequencies of resistant subjects exposed to aero-pollutants and OCOPD subjects exposed to aero-pollutants were compared according to the presence of 0, 1, and 2+ protective genotypes selected from a subset of 6 protective polymorphisms (COX2 −765 CC/CG, NOS3 298 TT, α1AT MS/SS, SOD3 AG/GG, MEH Exon 3 RR, VDBP 420 AA), significant differences were found (see Table 16). 63% of those with 0 protective polymorphisms were OCOPD sufferers, compared to only 42% of those with 2+ protective polymorphisms, with Odd's ratio of about 2.

From the analyses of the individual polymorphisms, 11 susceptibility genotypes were identified and analyzed for their frequencies in the smoker cohort consisting of resistant smokers and those with OCOPD. When the frequencies of resistant subjects exposed to aero-pollutants and OCOPD subjects exposed to aero-pollutants were compared according to the presence of 0, 1 and 2+ susceptibility genotypes selected from a subset of 5 of the susceptibility polymorphisms (MMP1 −1607 2G2G, GSTP1 105 GG, PAI-1 −675 5G5G, IL-13 −1055 TT, VDBP 416 GG), significant differences were found (see Table 17). 73% of those with 2+ susceptibility polymorphisms were OCOPD, compared with only 45% of those with 0 susceptibility polymorphisms, with Odd's ratio of about 2.

These findings indicate that the methods of the present invention can be predictive of OCOPD in an individual well before symptoms present.

These findings therefore also present opportunities for therapeutic interventions and/or treatment regimens, as discussed herein. Briefly, such interventions or regimens can include the provision to the subject of motivation to implement a lifestyle and/or occupational change, or therapeutic methods directed at normalising aberrant gene expression or gene product function. For example, the −765 G allele in the promoter of the gene encoding COX2 is associated with increased expression of the gene relative to that observed with the C allele. As shown herein, the C allele is protective with respect to predisposition to risk of developing OCOPD, whereby a suitable therapy in subjects known to possess the −765 G allele can be the administration of an agent capable of reducing expression of the gene encoding COX2.

### EXAMPLE 23

A subject with the −765G allele is identified, as described above. Following this, an agent capable of reducing the function of the gene encoding COX2, or the activity of COX2, is administered to the subject. An alternative suitable therapy can be the administration to such a subject of a COX2 inhibitor such as additional therapeutic approaches, gene therapy, RNAi. Thus, the susceptibility of the subject to developing OCOPD will be decreased.

As shown herein the −133 C allele in the promoter of the gene encoding IL18 is associated with susceptibility to OCOPD. The −133 G allele in the promoter of the gene encoding IL18 is associated with increased IL18 levels, whereby a suitable therapy in subjects known to possess the −133 C allele can be the administration of an agent capable of increasing expression of the gene encoding IL18.

### EXAMPLE 24

A subject with the −133C allele in the promoter of the gene encoding IL18 will be identified as described above. An agent capable of increasing expression of the gene encoding IL18 will be provided to the subject (for example, additional IL18). Repeated doses will be administered as needed. Thus, the susceptibility of the subject to developing OCOPD will be decreased.

As shown herein the −675 5G5G genotype in the promoter of the plasminogen activator inhibitor gene is associated with susceptibility to OCOPD. The 5G allele is reportedly associated with increased binding of a repressor protein and decreased transcription of the gene. A suitable therapy can be the administration of an agent capable of decreasing the level of repressor and/or preventing binding of the repressor, thereby alleviating its downregulatory effect on transcription. An alternative therapy can include gene therapy, for example the introduction of at least one additional copy of the plasminogen activator inhibitor gene having a reduced affinity for repressor binding (for example, a gene copy having a −675 4G4G genotype).

### EXAMPLE 25

A subject with the −675 5G5G genotype is identified, as described above. The subject is administered an agent capable of preventing the binding of the repressor (for example, an antibody to the repressor), thereby alleviating the repressor's downregulatory effect on transcription. Doses are repeated as necessary to reduce the effect of the SNP. Thus, the susceptibility of the subject to developing OCOPD will be decreased.

An alternative therapy can include gene therapy, for example the introduction of at least one additional copy of the plasminogen activator inhibitor gene having a reduced affinity for repressor binding (for example, a gene copy having a −675 4G4G genotype).

### EXAMPLE 26

A subject with the AA genotype at the 874 A/T polymorphism in the gene encoding interferon-γ is identified. The subject is administered an agent capable of modulating interferon-γ activity, thereby restoring the subject's interferon-γ activity to a normal (for an individual not at risk of developing OCOPD due to this SNP) level. Doses of the agent are repeated as necessary to reduce the effect of the SNP. Thus, the susceptibility of the subject to developing OCOPD will be decreased.

### EXAMPLE 27

A subject with the CC genotype at the −159 C/T polymorphism in the gene encoding CD-14 is identified. The subject is administered an agent capable of modulating CD-14 and/or IgE activity, thereby restoring the subject's CD-14 activity to a normal (for an individual not at risk of developing OCOPD due to this SNP) level. Doses of the agent are repeated as necessary to reduce the effect of the SNP. Thus, the susceptibility of the subject to developing OCOPD will be decreased.

In some of the above embodiments, the subject having an increased risk of developing OCOPD is identified by the presence of a particular genetic marker. In other situations, the subject can be identified by their environmental conditions. Suitable methods and agents for use in such therapy are well known in the art, and are discussed herein.

The identification of both susceptibility and protective polymorphisms as described herein also provides the opportunity to screen candidate compounds to assess their efficacy in methods of prophylactic and/or therapeutic treatment. Such screening methods involve identifying which of a range of candidate compounds have the ability to reverse or counteract a genotypic or phenotypic effect of a susceptibility polymorphism, or the ability to mimic or replicate a genotypic or phenotypic effect of a protective polymorphism.

### EXAMPLE 28

The present example provides one example for how one can screen for compounds that modulate the expression of a gene whose expression is upregulated or downregulated when associated with a susceptibility or protective polymorphism.

One first obtains a cell that includes a particular gene of interest. The expression of the gene of interest is upregulated or downregulated when associated with a susceptibility or a protective polymorphism. The polymorphism (and gene) can be selected from the following: −765 CC or CG in the promoter of the gene encoding COX2, −251 AA genotype in the promoter of the gene encoding IL-8, Lys 420 Thr AA genotype in the gene encoding VDBP, Glu 416 Asp TT or TG genotype in the gene encoding VDBP, exon 3 T/C RR genotype in the gene encoding MEH, Arg 312 Gln AG or GG genotype in the gene encoding SOD3, MS or SS genotype in the gene encoding α1AT, Asp 299 Gly AG or GG genotype in the gene encoding TLR4, Gln 27 Glu CC genotype in the gene encoding ADRB2, −518 AA genotype in the gene encoding IL-11, Asp 298 Glu TT genotype in the gene encoding NOS3, −765 GG in the promoter of the gene encoding COX2, Ile 105 Val GG in the gene encoding GSTP1, 105 AA in the gene encoding IL-18, −133 CC in the promoter of the gene encoding IL-18, Lys 420 Thr CC in the gene encoding VDBP, Glu 416 Asp GG in the gene encoding VDBP, Arg 312 Gln AA in the gene encoding SOD3, 3′ 1237 Tt or tt in the gene encoding α1-Antitrypsin, −1055 TT in the promoter of the gene encoding IL-13, −675 5G5G in the promoter of the gene encoding PAI-1, and −1607 2G2G in the gene encoding MMP1. In the cell, the expression of the gene is neither upregulated nor down-regulated except as noted below.

One then contacts the cell expressing the gene of interest with the candidate compound. Next, one determines the level of expression of the gene following contact of the cell with the candidate compound. A change in the level of expression after the contacting step as compared to before the contacting step is indicative of the ability of the compound to modulate an expression, activity, or expression and activity of the gene.

Still further, methods for assessing the likely responsiveness of a subject to an available prophylactic or therapeutic approach are provided. Such methods have particular application where the available treatment approach involves restoring the physiologically active concentration of a product of an expressed gene from either an excess or deficit to be within a range which is normal for the age and sex of the subject. In such cases, the method includes the detection of the presence or absence of a susceptibility polymorphism which when present either upregulates or downregulates expression of the gene such that a state of such excess or deficit is the outcome, with those subjects in which the polymorphism is present being likely responders to treatment.

### EXAMPLE 29

This example demonstrates one method of estimating the potential responsiveness of a subject to a prophylactic or therapeutic treatment for OCOPD. The treatment involves restoring the physiologically active concentration of a product of gene expression to within a range that is normal for the age and sex of a subject without OCOPD.

A subject at risk of developing OCOPD is first identified. Next, one detects the presence or absence of a susceptibility polymorphism in the subject. The susceptibility polymorphism can be selected from the group consisting of: −765 GG in the promoter of the gene encoding COX2, Ile 105 Val GG in the gene encoding GSTP 1, 105 AA in the gene encoding IL-18, −133 CC in the promoter of the gene encoding IL-18, Lys 420 Thr CC in the gene encoding VDBP, Glu 416 Asp GG in the gene encoding VDBP, Arg 312 Gln AA in the gene encoding SOD3, 3′ 1237 Tt or tt in the gene encoding α1-Antitrypsin, −1055 TT in the promoter of the gene encoding IL-13, −675 5G5G in the promoter of the gene encoding PAI-1, and −1607 2G2G in the gene encoding MMP1, or any combination thereof. The presence of the polymorphism in the subject indicates that the subject will respond to treatment with an agent that appropriately alters the expression or activity of the particular gene that is altered by the polymorphism.

### EXAMPLE 30

Table 21 below presents representative examples of polymorphisms in linkage disequilibrium with the polymorphisms specified herein. Examples of such polymorphisms can be located using public databases, such as that available at world wide web “dot” Hapmap “dot” org. Specified polymorphisms are indicated in parentheses.

TABLE 21
Polymorphism Reported To Be In LD With
Polymorphisms Specified Herein.
COX2

| | | | | |
|---|---|---|---|---|
| rs7527769 | rs689465 | rs4648270 | rs7550380 | rs12027712 |
| rs12759220 | rs2206594 | rs689466 | rs20430 | rs6687495 |
| rs2745558 | rs4648271 | rs6681231 | rs3918304 | rs11567825 |
| rs13376484 | rs20415 | rs4648273 | rs12064238 | rs20416 |
| rs16825748 | rs10911911 | rs4648254 | rs4648274 | rs12743673 |
| rs11567815 | rs16825745 | rs10911910 | rs20417 | rs20432 |
| | | | (−765G > C) | |
| rs12743516 | rs20433 | rs10911909 | rs4648256 | rs3218622 |
| rs1119066 | rs20419 | rs2066826 | rs1119065 | rs2734779 |
| rs5278 | rs1119064 | rs20420 | rs4648276 | rs10798053 |
| rs20422 | rs20434 | rs12409744 | rs20423 | rs3218623 |
| rs10911908 | rs5270 | rs3218624 | rs10911907 | rs20424 |
| rs5279 | rs7416022 | rs5271 | rs4648278 | rs2745561 |
| rs4648257 | rs13306034 | rs10911906 | rs11567819 | rs2853803 |
| rs2734776 | rs3134591 | rs4648279 | rs2734777 | rs3134592 |
| rs4648281 | rs12084433 | rs20426 | rs4648282 | rs2734778 |
| rs4648258 | rs11567826 | rs2745560 | rs11567820 | rs4648283 |
| rs2223627 | rs2745557 | rs4648284 | rs2383517 | rs11567821 |
| rs4648285 | rs4295848 | rs4648259 | rs11567827 | rs4428839 |
| rs4648260 | rs4648286 | rs4609389 | rs4648261 | rs4648287 |
| rs4428838 | rs4648262 | rs5272 | rs12131210 | rs11567822 |
| rs4648288 | rs2179555 | rs11567823 | rs5273 | rs2143417 |
| rs2066824 | rs5274 | rs2143416 | rs20427 | rs3218625 |
| rs11583191 | rs5277 | rs4648289 | rs2383516 | rs2066823 |
| rs4648290 | rs2383515 | rs4648263 | rs1051896 | rs10911905 |
| rs4987012 | rs5275 | rs10911904 | rs20428 | rs6684912 |
| rs20429 | rs2745559 | rs4648264 | rs12042763 | rs4648265 |
| rs4648250 | rs4648266 | rs4648251 | rs4648267 | rs2223626 |
| rs11567824 | rs689462 | rs4648268 | rs4648253 | rs4648269 |

ADRB2

| | | | | |
|---|---|---|---|---|
| rs2082382 | rs2053044 | rs1126871 | rs2082394 | rs17108803 |
| rs6885272 | rs2082395 | rs12654778 | rs6889528 | rs9325119 |
| rs11168070 | rs4521458 | rs9325120 | rs11959427 | rs10463409 |
| rs12189018 | rs1042711 | rs7702861 | rs11168066 | rs1801704 |
| rs11959615 | rs1042713 | rs11958940 | rs4705270 | rs1042714 |
| | (Arg 16 Gly) | | | (Gln 27 Glu) |
| rs10079142 | rs9325121 | rs1042717 | rs11746634 | rs1800888 |
| rs11168067 | rs1042718 | rs9325122 | rs3729943 | rs11957351 |
| rs1042719 | rs11948371 | rs3729944 | rs11960649 | rs3730182 |
| rs1432622 | rs1042720 | rs1432623 | rs6879202 | rs11168068 |
| rs3777124 | rs17778257 | rs1803051 | rs2400706 | rs8192451 |
| rs2895795 | rs4987255 | rs2400707 | rs3177007 | |

IL18

| | | | | |
|---|---|---|---|---|
| rs187238 | rs5744238 | rs5744255 | rs5744228 | rs5744239 |
| rs5744256 | rs360718 | rs7932965 | rs5744257 | rs360717 |
| rs11214103 | rs360720 | rs5744229 | rs5744241 | rs5744258 |
| rs100000353 | rs5744242 | rs5744259 | rs5744231 | rs5744243 |
| rs5744260 | rs5744232 | rs5744244 | rs5744261 | rs7106524 |
| rs360722 | rs549908 | rs189667 | rs5023207 | rs12290658 |
| | (105 A/C) | | | |
| rs5744246 | rs12271175 | rs5744247 | rs11606049 | rs360721 |
| | | | | (−133 C/G) |
| rs360716 | rs360715 | rs4988359 | rs360714 | rs12721559 |
| rs2043055 | rs5744248 | rs5744233 | rs5744249 | rs795467 |
| rs5744250 | rs12270240 | rs5744251 | rs100000354 | rs100000356 |
| rs4937113 | rs1834481 | rs100000355 | rs17215057 | rs360723 |
| rs5744253 | rs5744237 | rs5744254 | | |

PAI1

| | | | | |
|---|---|---|---|---|
| rs6465787 | rs2854226 | rs7788533 | rs2227707 | rs6975620 |
| rs2227631 | rs6956010 | No rs number | rs12534508 | rs4729664 |
| | | (−675 4G/5G) | | |
| rs2527316 | rs2854235 | rs10228765 | rs2854225 | |

NOS3

| | | | | |
|---|---|---|---|---|
| rs2373962 | rs3918225 | rs3918169 | rs2373961 | rs3918160 |
| rs3918170 | rs6951150 | rs1800779 | rs3793342 | rs13238512 |
| rs2243311 | rs3793341 | rs10247107 | rs3918161 | rs1549758 |
| rs10276930 | rs10952298 | rs1007311 | rs10277237 | rs2070744 |
| | | | | (−786 T/C) |
| rs9282803 | rs12703107 | rs9282804 | rs6946340 | rs3918226 |
| rs1799983 | rs6946091 | rs3918162 | rs6946415 | rs3918163 |
| (Asp 298 Glu) | | | | |
| rs10952296 | rs3918164 | rs13309715 | rs3918165 | rs10952297 |
| rs1800781 | rs7784943 | rs13310854 | rs11771443 | rs13310763 |
| rs2243310 | rs2853797 | rs1800783 | rs13311166 | rs3918155 |
| rs13310774 | rs3918156 | rs2853798 | rs2566519 | rs11974098 |
| rs3918157 | rs3918166 | rs3918158 | rs3730001 | rs3918159 |
| rs3918167 | rs2566516 | rs3918168 | | |

VDBR

| | | | | |
|---|---|---|---|---|
| rs222035 | rs844806 | rs705117 | rs222036 | rs1491709 |
| rs2070741 | rs16846943 | rs705119 | rs2070742 | rs7668653 |
| rs6845925 | rs6821541 | rs1491720 | rs12640255 | rs222048 |
| rs16845007 | rs12644050 | rs432031 | rs17830803 | rs6845869 |
| rs432035 | rs7041 | rs12640179 | rs222049 | rs222042 |
| | (Glu 416 Asp) | | | |
| rs222050 | rs4588 | rs3187319 | rs12510584 | rs222043 |
| | (Lys 420 Thr) | | | |
| rs17467825 | rs3737553 | rs842999 | rs9016 | rs222044 |
| rs1352846 | rs222045 | rs222039 | rs16846912 | rs3775154 |
| rs222046 | rs222040 | rs705118 | rs843005 | rs222047 |
| rs222041 | rs13142062 | rs7672977 | rs843000 | rs705121 |
| rs3755967 | rs11723621 | rs1491710 | rs2298850 | rs2282678 |
| rs705120 | rs2282679 | rs2298851 | rs2282680 | |

GSTP1

| | | | | |
|---|---|---|---|---|
| rs656652 | rs6591255 | rs762803 | rs625978 | rs8191430 |
| rs8191449 | rs6591251 | rs6591256 | rs947894 | rs12278098 |
| | | | (Ile 105 Val) | |
| rs8191431 | rs612020 | rs8191432 | rs4986948 | rs12284337 |
| rs7109914 | rs675554 | rs12574108 | rs4147580 | rs749174 |
| rs6591252 | rs8191436 | rs8191450 | rs597717 | rs8191437 |
| rs743679 | rs688489 | rs17593068 | rs1799811 | rs597297 |
| rs8191438 | rs11553890 | rs6591253 | rs8191439 | rs4986949 |
| rs6591254 | rs8191440 | rs8191451 | rs7927381 | rs8191441 |
| rs1871042 | rs7940813 | rs1079719 | rs11553892 | rs593055 |
| rs1871041 | rs4891 | rs7927657 | rs4147581 | rs6413486 |
| rs614080 | rs8191444 | rs5031031 | rs7941395 | rs8191445 |
| rs947895 | rs7941648 | rs2370143 | rs7945035 | rs8191446 |
| rs2370141 | rs3891249 | rs2370142 | rs8191447 | rs7949394 |
| rs12796085 | rs7949587 | rs8191448 | | |

SOD3

| | |
|---|---|
| rs1799895 (Arg 213 Gly) | Region of low LD |

alpha1 antitrypsin

| | | | | |
|---|---|---|---|---|
| rs709932 | rs2749521 | rs877081 | rs11558261 | rs2753939 |
| rs877082 | rs20546 | rs1802959 | rs877083 | rs11558263 |
| rs1802961 | rs877084 | rs1028580 | rs1050469 | rs875989 |
| rs7145770 | Z allele, | rs9944117 | rs2239652 | rs1050520 |
| | no rs number | | | |
| rs1884546 | rs7145047 | rs12077 | rs1884547 | rs7141735 |
| rs12233 | rs1885065 | rs11558264 | rs13170 | rs1884548 |
| rs6647 | rs1303 | rs1243167 | rs8350 | rs1802960 |
| rs17751614 | rs2230075 | rs1243163 | rs1884549 | rs1049800 |
| rs2073333 | rs1243168 | rs17580 | rs1243164 | rs17090693 |
| | | (S allele) | | |
| rs7144409 | rs17824597 | rs2854258 | rs7142803 | rs2753937 |
| rs1243165 | rs2749547 | rs1051052 | rs1243162 | rs1243166 |
| rs2753938 | rs11628917 | rs2070709 | rs11832 | rs17090719 |
| rs9944155 | rs11846959 | rs11568814 | rs1802962 | |
| | | (1237 G/A) | | |

TLR4

| | | | | |
|---|---|---|---|---|
| rs2149356 | rs5030713 | rs5030727 | rs5030714 | rs5030728 |
| rs4986790 | rs5030729 | rs100001066 | rs2770145 | rs5030710 |
| (Asp 299 Gly) | | | | |
| rs2770144 | rs5030711 | rs5031050 | rs5030712 | rs11536884 |
| rs16906079 | rs4986791 | | | |

IL8

| | | | | |
|---|---|---|---|---|
| rs4694635 | rs2227527 | rs2227543 | rs11730560 | rs11730284 |
| rs1957663 | rs7682639 | rs12420 | rs13106097 | rs11944402 |
| rs4694636 | rs2227529 | rs16849942 | rs4694178 | rs7658422 |
| rs16849925 | rs2227530 | rs3181685 | rs4694637 | rs11940656 |
| rs16849928 | rs2227531 | rs11733933 | rs11729759 | rs1951700 |
| rs11730667 | rs2227532 | rs2227544 | rs10938093 | rs1951699 |
| rs16849934 | rs2227534 | rs2227545 | rs13109377 | rs1957662 |
| rs4073 | rs2227550 | rs1951236 | rs16849938 | rs2227546 |
| (−251 A/T) | | | | |
| rs1951237 | rs6831816 | rs2227535 | rs1126647 | rs6446955 |
| rs2227517 | rs2227536 | rs11545234 | rs6446956 | rs2227518 |
| rs2227537 | rs2227548 | rs6446957 | rs2227519 | rs2227538 |
| rs10938092 | rs16849945 | rs2227520 | rs1803205 | rs13112910 |
| rs1951239 | rs2227521 | rs2227539 | rs13142454 | rs1951240 |
| rs2227522 | rs3756069 | rs11937527 | rs1957661 | rs2227523 |
| rs2227307 | rs12647924 | rs7674884 | rs2227524 | rs2227549 |
| rs13152254 | rs16849958 | rs2227525 | rs2227540 | rs13138765 |
| rs17202249 | rs2227526 | rs2227306 | rs13139170 | rs1951242 |

IL11

| | |
|---|---|
| rs4252546 (−518 G/A) | Region of low LD |

mEH

| | |
|---|---|
| rs1051740 (Tyr 113 | Region of low LD |
| His exon 3 T/C) | |

IL13

| | | | | |
|---|---|---|---|---|
| rs1800925 | rs2069747 | rs2069748 | rs11575055 | rs1295686 |
| (−1055 C/T) | | | | |
| rs2069755 | rs20541 | rs2069741 | rs2069742 | rs2069749 |
| | (Arg 130 Gln) | | | |
| rs2069743 | rs1295685 | rs2069756 | rs848 | rs3212142 |
| rs2069750 | rs2066960 | rs847 | rs1295687 | rs3212145 |
| rs2069744 | rs2069745 | rs2069746 | | |

MMP1

| | | | | |
|---|---|---|---|---|
| rs529381 | rs685265 | rs1144396 | rs7107224 | rs504875 |
| rs1155764 | rs526215 | rs534191 | rs12280880 | rs509332 |
| rs542603 | rs12283759 | rs574939 | rs2105581 | rs573764 |
| rs470206 | rs7102189 | rs533621 | rs575727 | rs1799750 |
| | | | | (−1607 G/GG) |
| rs552306 | rs634607 | rs470211 | rs12286876 | rs470146 |
| rs12285331 | rs2075847 | rs519806 | rs473509 | rs12283571 |
| rs498186 | rs2839969 | rs2000609 | rs7125865 | rs570662 |
| rs11225427 | rs484915 | rs470307 | rs2408490 | rs12279710 |

### INDUSTRIAL APPLICATION

The present invention is directed to methods for assessing a subject's risk of developing occupational chronic obstructive pulmonary disease (OCOPD). The methods can include the analysis of polymorphisms herein shown to be associated with increased or decreased risk of developing OCOPD or the analysis of results obtained from such an analysis. The use of polymorphisms herein shown to be associated with increased or decreased risk of developing OCOPD in the assessment of a subject's risk are also provided, as are nucleotide probes and primers, kits, and microarrays suitable for such assessment. Methods of treating subjects having the polymorphisms herein described are also provided. Methods for screening for compounds able to modulate the expression of genes associated with the polymorphisms herein described are also provided.

Additional information regarding the above methods and compositions can be found in U.S. patent application Ser. No. 10/479,525, filed Jun. 16, 2004; and PCT Application No. PCT/NZ02/00106, filed Jun. 5, 2002, which further designates New Zealand Application No. 512169, filed Jun. 5, 2001; New Zealand Application No. 513016, filed Jul. 17, 2001, and New Zealand Application No. 514275, filed Sep. 18, 2001, each of the foregoing is herein incorporated by reference in its entirety. Additional information can also be found in PCT application Nos. ______ and ______, filed May 10, 2006, entitled “Methods and Compsitions for Assessment of Pulmonary Function and Disorders” and “Methods of Analysis of Polymorphisms and Uses Thereof” respectively, having Agent Reference Nos. 542813JBM and 542814JBM respectively, each of the foregoing is herein incorporated by reference in its entirety. PCT Application Agent Reference No. 542813JBM claims priority to: NZ application No. 539934, filed May 10, 2005; NZ application No. 541935, filed Aug. 19, 2005; and JP application No. 2005-360523, filed Dec. 14, 2005, each of the foregoing is herein incorporated by reference in its entirety. PCT Application Agent Reference No. 542814JBM claims priority to: NZ application No. 540249, filed May 20, 2005; and NZ application No. 541842, filed Aug. 15, 2005, each of the foregoing is herein incorporated by reference in its entirety. Additional information can also be found in U.S. Pat. App. No. ______, filed May 10, 2006, entitled “Methods of Analysis of Polymorphisms and Uses Thereof,” and U.S. Pat. App. No. ______, filed May 10, 2006, entitled “Methods and Compositions for Assessment of Pulmonary Function and Disorders”, having attorney docket Nos; SGENZ.014AUS and SGENZ.013AUS respectively, each of the foregoing which is herein incorporated by reference in its entirety. Additional information can be found in U.S. Pat. App. No. ______, and PCT Application No: ______ both entitled “Methods and Composition for Assessment of Pulmonary Function and Disorders” filed on the same date as the present application, having attorney docket No. SGENZ.016AUS and 542844JBM respectively, herein incorporated by reference in their entireties. Further information can be found in PCT Application No: ______, having the same title, filed on even date herewith, having Agent Reference No: 542843JBM. Additional information can be found in New Zealand App. Nos. 540202, (filed May 19, 2005); 541389, (filed Jul. 20, 2005); 540203, (filed May 19, 2005); 541787, (filed Aug. 11, 2005); and 543297, (filed Oct. 28, 2005), all entitled “Methods and Compositions for Assessment of Pulmonary Function and Disorder,” each of the foregoing in the present paragraph is herein incorporated by reference in its entirety.

### PUBLICATIONS

1. Leigh et al. Chest 2002, 121, 264.
2. Moscato et al. Curr Opin Allergy and Clin Immunol 2003, 3 (2), 109.
3. Mayer et al. Respiration Physiology 2002, 128, 3.
4. Viegi et al. Curr Opin Allergy and Clin Immunol 2002, 2 (2), 115.
5. Hnizdo et al. Am J Epidemiol. 2002, 156, 738.
6. Sandford A J, et al., 1999. Z and S mutations of the α1-antitrypsin gene and the risk of chronic obstructive pulmonary disease. Am J Respir Cell Mol Biol. 20; 287-291.
7. Maniatis, T., Fritsch, E. F. and Sambrook, J., Molecular Cloning Manual. 1989.
8. Papafili A, et al., 2002. Common promoter variant in cyclooxygenase-2 represses gene expression. Arterioscler Thromb Vasc Biol. 20; 1631-1635.
9. Ukkola, O., Erkkilä, P. H., Savolainen, M. J. & Kesäniemi, Y. A. 2001. Lack of association between polymorphisms of catalase, copper zinc superoxide dismutase (SOD), extracellular SOD and endothelial nitric oxide synthase genes and macroangiopathy in patients with type 2 diabetes mellitus. J Int Med 249; 451-459.
10. Smith CAD & Harrison D J, 1997. Association between polymorphism in gene for microsomal epoxide hydrolase and susceptibility to emphysema. Lancet. 350; 630-633.
11. Lorenz E, et al., 2001. Determination of the TLR4 genotype using allele-specific PRC. Biotechniques. 31; 22-24.

All patents, publications, scientific articles, and other documents and materials referenced or mentioned herein are indicative of the levels of skill of those skilled in the art to which the invention pertains, and each such referenced document and material is hereby incorporated by reference to the same extent as if it had been incorporated by reference in its entirety individually or set forth herein in its entirety. Any and all materials and information from the above patents, publications, scientific articles, web sites, electronically available information, and other referenced materials or documents can be physically incorporated into this specification.

The specific methods and compositions described herein are representative of various embodiments or preferred embodiments and are exemplary only and not intended as limitations on the scope of the invention. Other objects, aspects, examples and embodiments will occur to those skilled in the art upon consideration of this specification, and are encompassed within the spirit of the invention as defined by the scope of the claims. It will be readily apparent to one skilled in the art that varying substitutions and modifications can be made to the invention disclosed herein without departing from the scope and spirit of the invention. The invention illustratively described herein suitably can be practiced in the absence of any element or elements, or limitation or limitations, which is not specifically disclosed herein as essential. Thus, for example, in each instance herein, in embodiments or examples of the present invention, any of the terms “comprising”, “consisting essentially of”, and “consisting of” can be replaced with either of the other two terms in the specification, thus indicating additional examples, having different scope, of various alternative embodiments of the invention. Also, the terms “comprising”, “including”, containing”, etc. are to be read expansively and without limitation. The methods and processes illustratively described herein suitably can be practiced in differing orders of steps, and that they are not necessarily restricted to the orders of steps indicated herein or in the claims. It is also that as used herein and in the appended claims, the singular forms “a,” “an,” and “the” include plural reference unless the context clearly dictates otherwise. Thus, for example, a reference to “a host cell” includes a plurality (for example, a culture or population) of such host cells, and so forth. Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing during the prosecution of the application.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed can be resorted to by those skilled in the art, and that such modifications and variations are considered to be within the scope of this invention as defined by the appended claims.

1. A method of determining a subject's risk of developing occupational chronic obstructive pulmonary disease (OCOPD) comprising analyzing a sample from said subject for the presence or absence of one or more polymorphisms selected from the group consisting of:
−765 C/G in the promoter of the gene encoding Cyclooxygenase 2;
Ile 105 Val (A/G) in the gene encoding Glutathione S transferase P;
105 C/A in the gene encoding Interleukin-18;
−133 G/C in the promoter of the gene encoding Interleukin-18;
−251 A/T in the gene encoding Interleukin-8;
Lys 420 Thr (A/C) in the gene encoding Vitamin D binding protein;
Glu 416 Asp (T/G) in the gene encoding Vitamin D binding protein;
exon 3 T/C (R/r) in the gene encoding Microsomal epoxide hydrolase;
Arg 312 Gln (AC) in the gene encoding Superoxide dismutase 3;
1237 G/A (T/t) in the gene encoding α1-Antitrypsin;
α1-Antitrypsin (α1AT) S polymorphism;
Asp 299 Gly A/G in the gene encoding Toll-like receptor 4;
Gln27Glu in the gene encoding β2 Adrenoreceptor;
−518 G/A in the promoter of the gene encoding Interleukin-11;
−1055 (C/T) in the promoter of the gene encoding Interleukin-13;
−675 4G/5G in the promoter of the gene encoding Plasminogen activator inhibitor 1;
298 Asp/Glu (T/G) in the gene encoding Nitric oxide synthase 3;
−1607 1G/2G in the gene encoding Matrix metalloproteinase 1; and
one or more polymorphisms which are in linkage disequilibrium with any one or more of these polymorphisms;
wherein the presence or absence of one or more of said polymorphisms is indicative of the subject's risk of developing occupational chronic obstructive pulmonary disease.

2. A method according to claim 1 wherein the presence of one or more of the polymorphisms selected from the group consisting of:
−765 CC or CG in the promoter of the gene encoding COX2;
−251 AA genotype in the promoter of the gene encoding IL-8;
Lys 420 Thr AA genotype in the gene encoding VDBP;
Glu 416 Asp TT or TG genotype in the gene encoding VDBP;
exon 3 T/C RR genotype in the gene encoding MEH;
Arg 312 Gln AG or GG genotype in the gene encoding SOD3;
MS or SS genotype in the gene encoding α1AT;
Asp 299 Gly AG or GG genotype in the gene encoding TLR4;
Gln 27 Glu CC genotype in the gene encoding ADRB2;
−518 AA genotype in the gene encoding IL-11; and
Asp 298 Glu TT genotype in the gene encoding NOS3;
is indicative of a reduced risk of developing occupational chronic obstructive pulmonary disease.

3. A method according to claim 1 wherein the presence of one or more of the polymorphisms selected from the group consisting of:
−765 GG in the promoter of the gene encoding COX2;
Ile 105 Val GG in the gene encoding GSTP1;
105 AA in the gene encoding IL-18;
−133 CC in the promoter of the gene encoding IL-18;
Lys 420 Thr CC in the gene encoding VDBP;
Glu 416 Asp GG in the gene encoding VDBP;
Arg 312 Gln AA in the gene encoding SOD3;
3′1237 Tt or tt in the gene encoding α1-Antitrypsin;
−1055 TT in the promoter of the gene encoding IL-13;
−675 5G5G in the promoter of the gene encoding PAI-1; and
−1607 2G2G in the gene encoding MMP1;
is indicative of an increased risk of developing occupational chronic obstructive pulmonary disease.

4. A method according to claim 1 wherein the method further comprises analyzing said sample for the presence or absence of one or more further polymorphisms selected from the group consisting of:
M1 null in the gene encoding GST-1;
−82 A/G in the promoter of the gene encoding MMP12;
−1562 C/T within the promoter of the gene encoding MMP9;
T→C within codon 10 of the gene encoding TGFβ;
−1296 T/C within the promoter of the gene encoding TIMP3; and
one or more polymorphisms which are in linkage disequilibrium with one or more of these polymorphisms.

5. A method according to claim 4 wherein the presence of one or more of the polymorphisms selected from the group consisting of:
−1296TT within the promoter of the gene encoding TIMP3; and
CC (homozygous P allele) within codon 10 of the gene encoding TGFβ;
is indicative of a reduced risk of developing occupational chronic obstructive pulmonary disease.

6. A method according to claim 4 wherein the presence of one or more of the polymorphisms selected from the group consisting of:
−82AA within the promoter of the gene encoding MMP12; and
−1562CT or −1562TT within the promoter of the gene encoding MMP9;
is indicative of an increased risk of developing occupational chronic obstructive pulmonary disease.

7. A method of assessing a subject's risk of developing occupational chronic obstructive pulmonary disease, said method comprising the steps of:
(i) determining a presence or absence of at least one protective polymorphism associated with a reduced risk of developing occupational chronic obstructive pulmonary disease; and
(ii) in the absence of at least one protective polymorphism, determining a presence or absence of at least one susceptibility polymorphism associated with an increased risk of developing occupational chronic obstructive pulmonary disease,
wherein the presence of one or more of said protective polymorphism is indicative of a reduced risk of developing occupational chronic obstructive pulmonary disease, and wherein the absence of at least one protective polymorphism in combination with the presence of at least one susceptibility polymorphism is indicative of an increased risk of developing occupational chronic obstructive pulmonary disease.

8. (canceled)

9. (canceled)

10. (canceled)

11. (canceled)

12. The method of claim 7, wherein a presence of two or more protective polymorphims irrespective of the presence of one or more susceptibility polymorphisms is indicative of reduced risk of developing occupational chronic obstructive pulmonary disease.

13. The method of claim 7, wherein in the absence of a protective polymorphism the presence of one or more susceptibility polymorphisms is indicative of an increased risk of developing occupational chronic obstructive pulmonary disease.

14. The method of claim 7, wherein the presence of two or more susceptibility polymorphisms is indicative of an increased risk of developing occupational chronic obstructive pulmonary disease.

15. (canceled)

16. (canceled)

17. One or more nucleotide probes, primers, or primers and probes for use in the method of claim 1 wherein the one or more nucleotide probes, primers, or primers and probes span, or are able to be used to span, a polymorphic region of a gene in which the polymorphism to be analyzed is present.

18. A nucleic acid microarray which comprises a substrate presenting a nucleic acid sequence capable of hybridizing to a nucleic acid sequence that encodes one or more of the polymorphisms selected from the group defined in claim 1 or a sequence complimentary thereto.

19. (canceled)

20. (canceled)

21. (canceled)

22. A method of treating a subject having an increased risk of developing occupational chronic obstructive pulmonary disease comprising the step of replicating the presence, functional effect, or presence and functional effect of a protective polymorphism selected from the group defined in claim 2 in a subject, wherein said replicating is achieved genotypically, phenotypically, or both genotypically and phenotypically.

23. (canceled)

24. (canceled)

25. (canceled)

26. (canceled)

27. (canceled)

28. (canceled)

29. (canceled)

30. (canceled)

31. (canceled)

32. An antibody microarray comprising:
a substrate; and
an antibody presented on the substrate, wherein the antibody is capable of binding to a product of expression of a gene the expression of which is upregulated or downregulated when associated with a susceptibility or protective polymorphism selected from the group consisting of:
−765 CC or CG in the promoter of the gene encoding COX2;
−251 AA genotype in the promoter of the gene encoding IL-8;
Lys 420 Thr AA genotype in the gene encoding VDBP;
Glu 416 Asp TT or TG genotype in the gene encoding VDBP;
exon 3 T/C RR genotype in the gene encoding MEH;
Arg 312 Gln AG or GG genotype in the gene encoding SOD3;
MS or SS genotype in the gene encoding α1AT;
Asp 299 Gly AG or GG genotype in the gene encoding TLR4;
Gln 27 Glu CC genotype in the gene encoding ADRB2;
−518 AA genotype in the gene encoding IL-11;
Asp 298 Glu TT genotype in the gene encoding NOS3;
−765 GG in the promoter of the gene encoding COX2;
Ile 105 Val GG in the gene encoding GSTP1;
105 AA in the gene encoding IL-18;
−133 CC in the promoter of the gene encoding IL-18;
Lys 420 Thr CC in the gene encoding VDBP;
Glu 416 Asp GG in the gene encoding VDBP;
Arg 312 Gln AA in the gene encoding SOD3;
3′1237 Tt or tt in the gene encoding α1-Antitrypsin;
−1055 TT in the promoter of the gene encoding IL-13;
−675 5G5G in the promoter of the gene encoding PAI-1; and
−1607 2G2G in the gene encoding MMP1.

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. A kit for assessing a subject's risk of developing occupational chronic obstructive pulmonary disease, said kit comprising a means of analyzing a sample from said subject for a presence or an absence of one or more polymorphisms selected from the group consisting of:
−765 C/G in the promoter of the gene encoding Cyclooxygenase 2;
Ile 105 Val (A/G) in the gene encoding Glutathione S transferase P;
105 C/A in the gene encoding Interleukin-18;
−133 G/C in the promoter of the gene encoding Interleukin-18;
−251 A/T in the gene encoding Interleukin-8;
Lys 420 Thr (A/C) in the gene encoding Vitamin D binding protein;
Glu 416 Asp (T/G) in the gene encoding Vitamin D binding protein;
exon 3 T/C (R/r) in the gene encoding Microsomal epoxide hydrolase;
Arg 312 Gln (AC) in the gene encoding Superoxide dismutase 3;
3′ 1237 G/A (T/t) in the gene encoding α1-Antitrypsin;
α1-Antitrypsin (α1AT) S polymorphism;
Asp 299 Gly A/G in the gene encoding Toll-like receptor 4;
Gln27Glu in the gene encoding β2 Adrenoreceptor;
−518 G/A in the promoter of the gene encoding Interleukin-11;
−1055 (C/T) in the promoter of the gene encoding Interleukin-13;
−675 4G/5G in the promoter of the gene encoding Plasminogen activator inhibitor 1;
298 Asp/Glu (T/G) in the gene encoding Nitric oxide synthase 3;
−1607 1G/2G in the gene encoding Matrix metalloproteinase 1; and
one or more polymorphisms which are in linkage disequilibrium with any one or more of these polymorphisms.

47. The method of claim 1 further comprising analyzing the amino acid present at a position mapping to codon 420 of the gene encoding vitamin D binding protein.

48. The method of claim 47, wherein the presence of threonine at said position mapping to codon 420 of the gene encoding vitamin D binding protein is indicative of a predisposition to and/or potential risk of developing OCOPD, and/or potential onset of OCOPD.

49. The method of claim 47, wherein the presence of lysine at said position mapping to codon 420 of the gene encoding vitamin D binding protein is indicative of reduced risk of developing OCOPD and/or reduced potential onset of OCOPD.

50. The method of claim 1, further comprising analyzing an amino acid present at a position mapping to a codon selected from the group consisting of: 416 of a gene encoding VDBP: 312 of a gene encoding SOD3: codon 299 of a gene encoding TLR4; codon 27 of a gene encoding ADRB2; and codon 298 of a gene encoding nitric oxide synthase (NOS3).

51. (canceled)

52. (canceled)

53. (canceled)

54. (canceled)

55. The method of 50 wherein the presence of a glutamate at a position mapping to codon 298 of the gene encoding nitric oxide synthase is indicative of a predisposition to, potential risk of, or both predisposition and potential risk of developing OCOPD, potential onset of OCOPD, or developing and potential onset of OCOPD.

56. The method of 50, wherein the presence of an asparagine at a position mapping to codon 298 of the gene encoding nitric oxide synthase is indicative of reduced risk of developing OCOPD, reduced potential onset of OCOPD, and reduced risk of developing and reduced potential onset of OCOPD.

57. (canceled)
